(19) United States
(12) Patent Application Publication
(10) Pub. No.: US 2019/0292537 A1
(43) Pub. Date: Sep. 26, 2019

(54) Prokaryotic Argonaute Proteins and Uses Thereof

(71) Applicant: Wageningen Universiteit, Wageningen (NL)

(72) Inventors: John VAN DER OOST, Renkum (NL); Jorrit Wietze HEGGE, Wageningen (NL)

(21) Appl. No.: 16/317,078

(22) PCT Filed: Jul. 11, 2017
(86) PCT No.: PCT/EP2017/067462

§ 371 Date: Jan. 11, 2019

(30) Foreign Application Priority Data
Jul. 12, 2016 (GB) 1612090.9
Nov. 18, 2016 (GB) 1619551.3

(51) Int. Cl. C12N 15/11; C07K 14/33; C12N 9/22

(52) U.S. Cl. CPC C12N 15/11; C07K 14/33; C12N 2800/80; C12N 2310/14; C12N 9/22

(57) ABSTRACT

The invention relates to the field of genetic engineering tools, methods and techniques for nucleic acid, gene or genome editing. Specifically, the invention concerns prokaryotic Argonaute (pAgo) polypeptides having nuclease activity against target DNA when pAgo is complexed with a DNA guide. The invention also provides expression vectors comprising nucleic acids encoding said polypeptides as well as compositions and kits for, and methods of cleaving and editing target nucleic acids in a sequence-specific manner. The polypeptides, nucleic acids, expression vectors, compositions, kits and methods of the invention allow site-specific modifications of genetic material, whether isolated from cells in vitro, or within cells in situ and as such may usefully find application in many fields of biotechnology, including, for example, synthetic biology, gene therapy and agricultural or microbial biotechnology.

### THE FIELD OF THE INVENTION

The present invention relates to prokaryotic Argonaute (pAgo) proteins having nuclease activity against target DNA when pAgo is complexed with a DNA guide. Site-specificity may be adjusted by selection of a particular nucleotide sequence of the DNA guide. The invention also relates to the use of pAgo-guide complexes for site-specific modifications of genetic material, whether isolated from cells in vitro, or within cells in situ. The invention therefore, concerns pAgo proteins for use in gene editing techniques whereby the genome of a living cell is altered even down to the level of a single nucleotide base change.

### BACKGROUND TO THE INVENTION

Prokaryotic Argonautes (pAgos) are prokaryotic homologs of eukaryotic Argonaute proteins, which are known to be key enzymes in RNA interference pathways in which they complex with small RNA guides in RNA-induced silencing complexes (RISCs). In eukaryotes, RNA interference (RNAi) is a major mechanism of regulating endogenous gene expression and is also used in defence against viruses and transposable elements.

Argonaute proteins which function as endonucleases are known to form an evolutionarily conserved family comprising three key functional domains: (i) a carboxy-terminal PIWI (P-element Induced Wimpy Testis) endonuclease domain with a characteristic catalytic tetrad that will cleave a target nucleic acid, (ii) the MID domain which binds the 5′ phosphate and first nucleotide of the nucleic acid guide (a single-stranded RNA in eukaryotes), and (iii) the PAZ domain which uses its oligonucleotide-binding fold to secure the 3′ end of a guide strand. The PIWI domain resembles another nuclease, RNase H, a DNA-guided ribonuclease. Like RNase H, the PIWI domain contains four evolutionary conserved amino acids—typically aspartate-glutamate-aspartate-aspartate/histidine (DEDD/H)—that in a eukaryotic Argonaute endonuclease form a catalytic tetrad responsible for binding two Mg²⁺ ions and cleaving a target RNA into products bearing a 3′ hydroxyl and 5′ phosphate group (Nakanishi et al. (2012) Nature 486, 38-374). Unlike in the case of an RNase H, however, the guide strand, e.g. a small interfering RNA (siRNA), remains stably bound to the Argonaute protein through many rounds of target cleavage by means of anchorage of the 5′ phosphate in the MID domain. Endonucleolytic cleavage of the target occurs at a single phosphodiester bond. The structure of an Argonaute protein has been found to ensure that the bond cleaved always lies between the target nucleotides paired to the tenth and eleventh nucleotides (from the 5′ end) of a normal RNA guide; these nucleotides are commonly referred to as g10 and g11 for the guide and t10 and t11 for the target.

Eukaryotic Argonaute proteins typically bind 19-25 nucleotide siRNAs and 21-23 nucleotide microRNAs (miRNAs). Both siRNAs and miRNAs are cut from double-stranded RNA precursors by RNase III-like enzymes such as Dicer. The ‘seed sequence’ of a siRNA guide—nucleotides g2 to g7 or g2 to g8—appears to provide nearly all of the specificity for target binding. Argonaute proteins pre-organise the seed sequence into a one-stranded helix whose conformation makes it ready to pair with a complementary target. Argonaute proteins accomplish this by binding the negatively charged phosphodiester backbone of seed sequence nucleotides, displaying the edges of bases g2 to g8 so that they are ready to base pair with t2 to t8 of the target. For reviews of current information on Argonaute proteins, reference is made to: Cenik and Zamore (2011) Current Biology 21 (12) R446-R449; Jinek and Doudna (2009) Nature 457, 405-412; Ketting (2011) Dev. Cell 20, 148-161 and Swarts et al., (2014) Nature Struct. Mol. Biol. 21(9); 743-753.

Multiple pAgo proteins have been characterized recently. This has led to new insights regarding their evolution, role and mechanism. Studies of the pAgos of both Aquifex aeolicus and Thermus thermophilus have shown that target cleaving complexes are most effectively formed in vitro using single-stranded DNA guide rather than RNA. While as noted above eukaryotic Argonaute proteins utilize small RNA guides, the above characterised pAgos in vitro show a higher affinity for DNA guides. These observations contribute to the idea that pAgo DNA-guided target cleavage can occur for DNA and RNA targets, with a different DNA/RNA target cleavage capability in pAgos from different prokaryotes. As such pAgos are key players in the defence of their host genome against mobile genetic elements. Similar host defence proteins, like Cas9, have shown to be effective genome editing tools because one could specifically program a pAgo to bind DNA for specific, targeted cleavage. Discovering, characterizing and producing new genome editing tools has the potential to advance biotechnology. Indeed, pAgos have a number of potential advantages over other nucleases, such as Cas9, for genome editing applications, such as being smaller in size than Cas9, not requiring a target sequence to be immediately upstream of a protospacer adjacent motif (PAM) and guide sequences that are much smaller than the ones required by Cas9.

Structures of various ternary complexes of T. thermophilus pAgo (TtAgo) catalytic mutants with 5′-phosphorylated 21-nt DNA guide and complementary target RNAs of lengths 12-, 15- and 19-nt (Wang et al. (2009) Nature 461, 754-761). These studies support a two state model with duplex zippering beyond one turn of the helix requiring release of the 3′-end of the guide from the PAZ pocket. The catalytic activity of the RNase H fold of the PIWI domain is associated with residues D478, E512, D546 and D660. It has been further shown that two Mg²⁺ cations (A, B) can be positioned to facilitate RNA cleavage as observed for other RNase H-type nucleases with cation A assisting nucleophilic attack by positioning and activating a water molecule and cation B stabilizing the transition state and leaving group. Wang et al. (2009; ibid.) further report single-stranded target DNA cleavage by 21 nt DNA guided TtAgo. Target DNA cleavage occurred in the presence of Mg²⁺ or Mn⁺ ions, but not Ca²⁺ ions.

While such studies have provided much structural insight on mechanism of action of Argonaute proteins, the physiological role of pAgos remained uncertain. Strikingly, no homologs of other essential proteins from eukaryotic RNAi pathways (Dicer/Drosha, RdRP, accessory RISC proteins) have been detected in prokaryotic genomes. A comparative genomics study has revealed that 32% of the sequenced archaeal genomes and 9% of the sequenced bacterial genomes possess pAgos (Makarova et al. (2009) ibid.; Swarts et al. (2014b) ibid.). These studies have revealed that there is much variation among pAgos with respect to domain architecture: some resemble the eukaryotic Argonautes (either with an active site or without), some are truncated versions, and some are fusions with distinct (predicted) nuclease domains, or co-occur in the same operon as (predicted) nucleases.

TtAgo exhibits endonuclease activity at 75° C., can independently acquire and use a short DNA guide to attack and cleave strands of dsDNA plasmids (Swarts et al. (2014a) ibid.). In vivo and in vitro analyses indicated that TtAgo catalyzes DNA-guided DNA interference that is responsible for reducing plasmid transformation and plasmid proliferation efficiencies (Swarts et al. (2014a) ibid.).

In contrast, Rhodobacter sphaeroides pAgo (RsAgo) has a typical Argonaute domain architecture but does not possess the catalytic tetrad described above. Hence it is concluded to be catalytically inactive (Olovnikov et al. (2013) ibid.). The RsAgo system has been shown to target DNA, and to be part of a defence system against mobile genetic elements (viruses, plasmids, transposons) (Miyoshi et al. (2016) Nature Communications). Indeed, it was shown to use short RNA guides to attack complementary double stranded DNA targets (after which the target could be cleaved by an additional nuclease), i.e. RNA-guided DNA interference (Olovnikov et al. (2013) ibid.). Like many of these inactive pAgo variants, the gene that encodes RsAgo is clustered with a potential nuclease (Makarova et al. (2009) ibid).

Swarts et al., (2015) (Nucleic Acids Research, Vol 43 (10)) have recently reported characterisation of an Argonaute from the thermophilic archaeon Pyrococcus furiosus (PfAgo). The enzyme was shown to be a multi-turnover protein that operates to cleave either ssDNA or dsDNA. Catalysis was dependent on the presence of Mn²⁺ or Co²⁺ divalent cations. However, the PfAgo protein bound with ssRNA guide was unable to direct cleavage of ssDNA or ssRNA targets. The optimal activity of the enzyme is observed at temperature ranges between 87° C. and 99.9° C.

WO 2015/157534 discloses a characterisation of the Argonaute from thermophilic Marinitoga piezophila (MpAgo), which is able to cleave ssRNA and ssDNA using 5′ hydroxylated ssRNA guide strands at 60° C. MpAgo, however, is unable to cleave dsDNA targets.

Although thermophilic pAgos cleave target DNA, pAgos such as TtAgo, MpAgo and PfAgo function at high temperature ranges between 60-100° C. (Swarts et al, (2015) ibid.). The high temperatures required for optimal activity limit potential utility of these enzymes for in vivo and in vitro applications in molecular biology.

### SUMMARY OF THE INVENTION

The inventors have discovered a mesophilic prokaryotic Argonaute CbAgo (Argonaute from Clostridium butyricum).

Accordingly, the present invention provides a prokaryotic Argonaute (pAgo) comprising an amino acid sequence of SEQ ID NO:1 or a sequence of at least 50% identity therewith, having binding activity for a single stranded DNA (ssDNA) guide, and having nuclease activity for a target DNA, whereby when a ssDNA guide having substantial complementarity to the target DNA is bound to the pAgo to form a pAgo-guide complex, and when the pAgo-guide complex is associated with the target DNA, there is a site-specific cutting of the target DNA when single stranded, or nicking of the target DNA when double stranded.

In other aspects, the invention provides a pAgo comprising a polynucleotide sequence of SEQ ID NO: 9 or a sequence hybridisable thereto, preferably under stringent conditions, having binding activity for a ssDNA guide, and having nuclease activity for a target DNA, whereby when a ssDNA guide having substantial complementarity to the target DNA is bound to the pAgo to form a pAgo-guide complex, and when the pAgo-guide complex is associated with the target DNA, there is a site-specific cutting of the target DNA when single stranded, or nicking of the target DNA when double stranded.

In another aspect the invention provides, a pAgo comprising a PIWI domain having an amino acid sequence of SEQ ID NO: 3 or a sequence of at least 50% identity therewith, having binding activity for a ssDNA guide, and having nuclease activity for a target DNA, whereby when a ssDNA guide having substantial complementarity to the target DNA is bound to the pAgo to form a pAgo-guide complex, and when the pAgo-guide complex is associated with the target DNA, there is a site-specific cutting of the target DNA when single stranded, or nicking of the DNA target when double stranded.

The invention also provides a pAgo comprising an amino acid sequence of SEQ ID NO:1 or a sequence of at least 50% identity therewith, having binding activity for a ssDNA guide, and nuclease activity for a target DNA; whereby:
a) when a first pAgo is bound to a first ssDNA guide to form a first pAgo-guide complex
b) when a second pAgo is bound to a second ssDNA guide to form a second pAgo-guide complex; the first and second guides having substantial identity to opposed strands of the target,
c) and both first and second pAgo-guide complexes are associated with the target,
   there is cleavage of the double stranded target DNA.

In another aspect, the invention provides a pAgo comprising a PIWI domain having a polynucleotide sequence of residues 1584 to 2244 of SEQ ID NO: 9 or a sequence hybridisable thereto, preferably under stringent conditions, having binding activity for a ssDNA guide, and having nuclease activity for a target DNA, whereby when a ssDNA guide having substantial complementarity to the target DNA is bound to the pAgo to form a pAgo-guide complex, and when the pAgo-guide complex is associated with the target DNA, there is a site-specific cutting of the target DNA when single stranded, or nicking of the target DNA when double stranded.

The invention also provides a pAgo comprising a polynucleotide sequence of SEQ ID NO: 9 or a sequence hybridisable thereto, preferably under stringent conditions, having binding activity for a single stranded ssDNA guide, and nuclease activity for a target DNA; whereby: when a first pAgo is bound to a first ssDNA guide to form a first pAgo-guide complex when a second pAgo is bound to a second ssDNA guide to form a second pAgo-guide complex; the first and second guides having substantial identity to opposed strands of the target, and both first and second pAgo-guide complexes are associated with the target, there is cleavage of the double stranded target DNA.

In another aspect, the invention provides an in vitro method of cleaving a single stranded target DNA, or nicking a double stranded target DNA, comprising the steps of providing a pAgo as described herein, and a ssDNA guide, wherein the guide and the pAgo form a pAgo-guide complex; contacting the resulting pAgo-guide complex with the target DNA, the target comprising a nucleotide sequence substantially complementary to the guide sequence, wherein the pAgo-guide complex cleaves the single stranded target, or nicks the double stranded target DNA at a specific site.

An advantage of the pAgo of the invention is an effective “locking-in” of the ssDNA guide to the pAgo-guide complex, particularly when the pAgo is expressed in a cell in the presence of the guide. The guide would usually be co-expressed with the pAgo. This advantage means that once programmed with a guide for making a site-specific nick or cut of the target DNA, the pAgo-guide complex is unable to be reprogrammed to a new site in the target, whether by design or by accident. The implication is the pAgos of the invention provide a highly reliable gene editing tool.

The pAgo-guide complexes of the invention have further advantage in not having any additional activities that result in further modification of target DNAs, other than nicking or cutting. For example, no removal of nucleotides from a target DNA have been ascertained. This contrasts to other mesophilic pAgos (Gao et al., 2016, Nature Biotechnology).

Another advantage of a single pAgo-guide complex of the invention is its ability to make a site-specific nick in an ssDNA target. Therefore, by providing a single guide species to a pAgo of the invention, rather than forward and reverse guides, the possibility of site-specific nicking rather than cutting of dsDNA is made available.

The pAgos of the invention are preferably 748 amino acids long, but may be shorter or longer than this by a contiguous number of amino acids. This number of amino acids (shorter or longer) may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 234, 235, 236, 237, 238, 239, 240, 241, 242, 243, 244, 245, 246, 247 248, 249 or 250.

Therefore, included are functional fragments of pAgo proteins as herein defined which are less than full length (748 amino acids) but which retain guide complex formation and site specific nuclease activity for target DNA.

When first and second pAgos of the invention are employed they are preferably identical, but need not necessarily be so.

A ssDNA guide is substantially complementary to a target DNA, by which is meant that the guide is either exactly complementary to a target DNA sequence of same-length as the guide comprised in the target DNA, or there a number of mismatches, usually isolated, possibly contiguous. The number of mismatches may be 1, 2, 3, 4, or 5, for example.

A pair of pAgos can cleave a double stranded DNA that results in a blunt-end cut. In other embodiments a pair of pAgos can cleave a double stranded DNA that results in a staggered cut.

The pAgo may have at least 80%, preferably at least 90%, more preferably at least 95% amino acid sequence identity to SEQ ID NO: 1.

Optionally, a pAgo of the invention does not comprise an N-terminal OB-fold domain; wherein the OB-fold domain is an amino acid sequence of SEQ ID NO:2 or a sequence of at least 80% identity therewith. In certain embodiments, the pAgo of the invention may comprise an N-terminal OB-fold domain; wherein the OB-fold domain is an amino acid sequence of SEQ ID NO:2 or a sequence of at least 80% identity therewith.

Optionally, a pAgo of the invention further comprises a nuclear localisation sequence (NLS) on either the 5′ or 3′ terminus, or on both termini of a pAgo. It is further contemplated that a pAgo of the invention has multiple NLS's on the 5′ or 3′ terminus, or both termini.

In preferred embodiments a pAgo has nuclease activity that takes place at a temperature in the range 10 to 50° C., preferably 32 to 44° C. Advantageously and in a preferred aspect pAgos of the invention have nuclease activity at 37° C.

The ssDNA guides which form pAgo-complexes are preferably 10 to 50 nucleotides in length, more preferably 15 to 30 nucleotides in length, even more preferably 20 to 25 nucleotides in length, e.g. 21, 22 or 23 nucleotides in length.

Once formed, a pAgo-guide complex is preferably stable in that the ssDNA guide bound by pAgo is not displaceable by contacting with a subsequently provided polynucleotide.

In some embodiments, the target DNA is single stranded. In other embodiments the target DNA is double stranded. Other possible target DNA includes negatively supercoiled plasmids, nicked plasmids, linear fragments including linearised plasmids, genomic DNA or chromosomal DNA.

The guide or guides may be phosphorylated ssDNA. Guide or guides can comprise a terminal 5′-triphosphate.

The nuclease activity of pAgos of the invention preferably require the presence of at least one cation selected from Mn⁺⁺, Mg⁺⁺, Ca⁺⁺, Cu⁺⁺, Fe⁺⁺, Co⁺⁺, Zn⁺⁺ and Ni⁺⁺, or any combination thereof. A particularly preferred cation is Mn⁺⁺. The concentration ranges of the cations used may vary from about 2.5 μM to about 2000 μM. A particularly preferred range is from about 250 μM to about 2000 μM.

In another aspect, the invention provides an in vitro method of cleaving a single stranded target DNA, or nicking a double stranded target DNA, comprising the steps of providing a pAgo as defined herein, and a ssDNA guide, wherein the guide and the pAgo form a pAgo-guide complex; contacting the resulting pAgo-guide complex with the target DNA, the target DNA comprising a nucleotide sequence substantially complementary to the ssDNA guide sequence, wherein the pAgo-guide complex cleaves the single stranded target DNA, or nicks the double stranded target DNA at a specific site.

In another aspect, the invention provides a method of site-specific cleavage of a ssDNA or site specific nicking of a double stranded DNA in a cell, comprising the steps of: (i) combining a pAgo as defined herein with a ssDNA guide; wherein the pAgo and guide form a pAgo-guide complex; and introducing the pAgo-guide complex in to a cell; wherein the ssDNA guide sequence is substantially complementary to a sequence comprised in the target DNA.

In yet further aspects the invention provides an in vitro method of cleaving a double stranded target DNA; comprising the steps of:
a) providing a first pAgo as defined herein and a first ssDNA guide, wherein the guide and the pAgo form a first pAgo-guide complex;
b) providing a second pAgo as defined herein and a second ssDNA guide, wherein the guide and the pAgo form a second pAgo-guide complex; wherein the first and second ssDNA guides have substantial identity to opposed strands of the double stranded target DNA; and
c) contacting both pAgo-guide complexes with a target DNA, the target DNA comprising a sequence substantially complementary to the guide sequence, wherein the pAgo-guide complexes cleave the double stranded target DNA at a specific site.

In another aspect, the invention provides a method of site specific cleavage of a double stranded target DNA in a cell, comprising the steps of:
a) providing a pAgo as defined herein and a first ssDNA guide, wherein the guide and the pAgo form a first pAgo-guide complex; and
b) providing a second pAgo as defined herein and a second ssDNA guide, wherein the guide and the pAgo form a second pAgo-guide complex; first and second ssDNA guides having substantial identity to opposed strands of the double stranded target DNA; and
c) introducing the pAgo-guide complexes into a cell e.g. by transformation, transfection or transduction.

In preferred embodiments, the ssDNA guide sequences are substantially complementary to a DNA sequence comprised in the target DNA.

In another aspect the invention provides a method of site-specific modification of the genetic material of a cell through expression of a pAgo as defined herein in a cell in the presence of at least one exogenous ssDNA guide, wherein an expression vector comprising a polynucleotide sequence of the pAgo is introduced into the cell separately or simultaneously with the ssDNA guide.

In some embodiments, a method of site-specific modification occurs in a cell that is isolated and therefore in vitro. In other embodiments the methods of the invention may be performed on cells in situ; whether in a living tissue, organ or animal, including human. Such methods may involve a pAgo that are encoded on a first expression vector. In other such methods, a second expression vector may encode one or more additional pAgos. In certain embodiments, a method of the invention may involve a single expression vector that encodes all pAgos. Some methods of the invention may involve an expression vector that is comprised in a viral vector e.g. a retroviral or lentiviral vector.

Methods of the invention as defined herein may be performed in a prokaryotic cell. In other embodiments, a method may be performed in a eukaryotic cell. In yet further embodiments, a method as defined herein may be performed in an archaeal cell.

In methods of the invention which are used to modify genetic material, the method may further comprise the step of providing to the cell a double stranded polynucleotide, preferably double stranded DNA which inserts at the site of the double stranded break in the chromosomal DNA of the cell. Alternatively, such methods may introduce a mutation into the target DNA resulting in a recombinant DNA, with such methods comprising an additional step of introducing a donor template with the desired mutation; wherein the mutation is located in the seed region of the pAgo-guide complexes. In brief, an example of how the process would work is as follows:

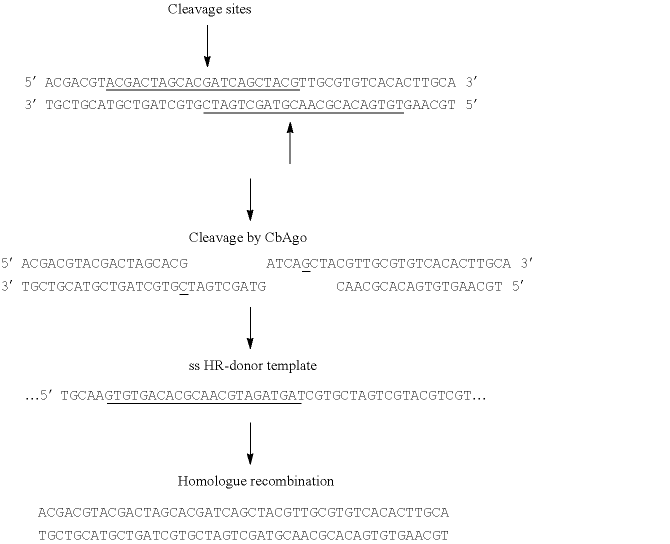

A method of the invention may further comprise making two spaced apart site-specific double stranded breaks that result in deletion of a DNA sequence bounded by the breaks.

The ssDNA guides, pAgo-guide complexes and target DNA are all as defined herein.

Methods of the invention performed in vitro, preferably employ an aqueous solution, ideally buffer, comprising at least one cation selected from Mn⁺⁺, Mg⁺⁺, Ca⁺⁺, Cu⁺⁺, Fe⁺⁺, Co⁺⁺, Zn⁺⁺ and Ni⁺⁺, or any combination thereof. A preferred cation is Mn⁺⁺.

The invention also provides a composition comprising at least one pAgo as defined herein, and at least one ssDNA guide. Compositions may further comprise a target DNA that comprises a nucleotide sequence that is substantially complementary to at least one DNA guide.

Additionally, the invention provides a polynucleotide encoding a pAgo as defined herein.

The invention further provides, an expression vector comprising a polynucleotide encoding a pAgo as defined herein.

The invention also provides a virus or viral vector comprising an expression vector as defined herein. In some embodiments, the virus or viral vector may be a retrovirus or lentivirus.

Also provided as aspects of the invention are kits. One kit comprises a pAgo defined herein, and at least one ssDNA guide. Another kit comprises an expression vector as defined herein and an ssDNA guide. Alternatively, a further kit comprises a virus or viral vector as defined herein, and a second virus or viral vector encoding an ssDNA guide.

In an alternative aspect, the invention provides a pAgo having an amino acid sequence of SEQ ID NO:1 or a sequence of at least 50% identity therewith, having binding activity for a ssDNA or ssRNA guide, and lacking nuclease activity for a target DNA or RNA, whereby when a ssDNA or ssRNA guide having substantial complementarity to the target DNA or RNA is bound to the pAgo to form a pAgo-guide complex, and when the pAgo-guide complex is associated with the target DNA or RNA, there is a site-specific blocking of a target DNA or RNA.

In a further alternative aspect, the invention provides a pAgo comprising a PIWI domain having an amino acid sequence of SEQ ID NO:3 or a sequence of at least 50% identity therewith, having binding activity for a ssDNA or ssRNA guide, and lacking nuclease activity for a target DNA or RNA, whereby when a ssDNA or ssRNA guide having substantial complementarity to the target DNA or RNA is bound to the pAgo to form a pAgo-guide complex, and when the pAgo-guide complex is associated with the target RNA or DNA, there is a site-specific blocking of a target RNA or DNA.

The absence of nuclease activity, particularly endonuclease activity, is provided by mutation in one or more of the amino acid residues of the pAgo protein essential for catalytic activity; that is to say in at least one of the four evolutionarily conserved amino acid tetrads (DEDD/H). So, for example, the mutation may be a single change of amino acid in any one or more of the following amino acid sequence portions of the pAgo protein:

CFIGL [symbol] VGTR
TIPQSG [symbol] KIAET
IVIHR [symbol] GFSRE
TTGYA [symbol] KICKA

More particularly, the amino acid change is a single change at one or more of the highlighted residues in the above. The single change is preferably a non-conservative substitution, so for example D to A, or E to A. Any substitution therefore is possible, other than D to E or E to D.

Instead of substitution, one or more of the highlighted residues can be simply deleted, optionally together with one or more amino acids within the sequence motif, contiguously or non-contiguously. One or more of the sequence motifs can in their entirety be deleted. Any combination of the above changes can be made, e.g. a non-conservative change in one motif and deletion of the other three motifs.

The structural features of the nuclease deficient pAgos of the invention may also include any of the structural variations as hereinbefore defined in relation to the nuclease active pAgos. So for example, the range of sequence identities compared to reference sequence, the composition of the pAgo in terms of amino acid domains, and overall lengths in terms of amino acids. Similarly with the guides, these are as defined in relation to the nuclease active pAgos of the invention.

The absence of endonuclease activity of the pAgo-guide complex in the aforementioned alternative aspects of the invention means that there is advantageously available a way of blocking a specific site in a target DNA or RNA, by way of specific sequence recognition, whether the target is a single or double stranded target. Such site-specific blocking provides for accurate means of blocking transcription of genes as may be desired, or blocking, disrupting or interfering with specific sites involved in regulation of gene expression.

The invention therefore further provides, an in vitro method of site-specific, targeted blocking of a target DNA or RNA, comprising the steps of: providing a nuclease inactive pAgo as defined herein, and a single stranded ssDNA or ssRNA guide, wherein the guide and the pAgo form a pAgo-guide complex; contacting the resulting pAgo-guide complex with the target DNA or RNA, wherein the target comprises a sequence substantially complementary to the guide sequence, wherein the pAgo-guide complex associates with the DNA or RNA at the site(s) of substantial complementarity between guide and target.

Additionally, the invention provides a method of site-specific blocking of a target polynucleotide in a cell, comprising contacting
a) a nuclease inactive pAgo as hereinbefore defined with a ssDNA guide, wherein the pAgo and guide form a pAgo-guide complex, and
b) introducing the pAgo-guide complex in to a cell, e.g. by transformation, transfection or microinjection, and wherein the guide sequence is substantially complementary to a DNA or RNA sequence comprised in the target DNA or RNA.

Also, the invention includes a method of site-specific, targeted blocking of a target DNA or RNA in a cell, comprising the steps of: transfecting, transforming or transducing the cell with an expression vector encoding (i) a nuclease inactive pAgo as hereinbefore defined, and transfecting, transforming or transducing (ii) a first ssRNA guide sequence, and (iii) a second ssRNA guide sequence; wherein at least one of the guide sequences is substantially complementary to a DNA or RNA sequence comprised in the target DNA or RNA, and wherein expression of the pAgo and guides in the cell results in pAgo-guide complexes which have site-specific blocking activity.

Advantageously the site-specific polynucleotide target blocking methods using the nuclease inactive pAgos of the invention allows for the targeted disruption of gene expression, and/or the targeted disruption of the control elements of gene expression, e.g. promotors or enhancers. In each of the aforementioned methods of site-specific blocking of target DNA or RNA, particular preferred or optional aspects of the methods are as defined herein in relation to the nuclease active pAgos of the invention.

In further aspects, the invention provides pAgo comprising an amino acid sequence of SEQ ID NO: 1 or a sequence of at least 50% identity therewith, having binding activity for a ssDNA guide, and having nuclease activity for a target DNA, wherein the guide is bound to the pAgo to form a pAgo-guide complex, and when the pAgo-guide complex is associated with the target DNA, there is cutting of the target DNA when single stranded, or nicking of the target DNA when double stranded.

In other aspects, the invention provides a pAgo comprising a polynucleotide sequence of SEQ ID NO. 9 or a sequence hybridisable thereto, preferably under stringent conditions, having binding activity for a ssDNA guide, and having nuclease activity for a target DNA, wherein the guide is bound to the pAgo to form a pAgo-guide complex, and when the pAgo-guide complex is associated with the target DNA, there is a cutting of the target DNA when single stranded, or nicking of the target DNA when double stranded.

In another aspect, the invention pAgo comprising a PIWI domain having an amino acid sequence of SEQ ID NO:3 or a sequence of at least 50% identity therewith, having binding activity for a ssDNA guide, and having nuclease activity for a target DNA, whereby when a ssDNA guide is bound to the pAgo to form a pAgo-guide complex, and when the pAgo-guide complex is associated with the target DNA, there is cutting of the target DNA when single stranded, or nicking of the target DNA when double stranded.

The invention also provides a pAgo comprising an amino acid sequence of SEQ ID NO: 1 or a sequence of at least 50% identity therewith, having binding activity for an ssDNA guide, and nuclease activity for a target DNA; whereby:
a) when a first pAgo is bound to a first ssDNA guide to form a first pAgo-guide complex
b) when a second pAgo is bound to a second ssDNA guide to form a second pAgo-guide complex;
c) and both first and second pAgo-guide complexes are associated with the target,
   and there is cleavage of the double stranded target DNA.

In another aspect, the invention provides a pAgo comprising a PIWI domain having a polynucleotide sequence of residues 1584 to 2244 of SEQ ID NO:9 or a sequence hybridisable thereto, preferably under stringent conditions, having binding activity for a ssDNA guide, and having nuclease activity for a target DNA, whereby when a ssDNA guide is bound to the pAgo to form a pAgo-guide complex, and when the pAgo-guide complex is associated with a target DNA, there is cutting of the target DNA when single stranded, or nicking of the target DNA when double stranded.

The invention also provides a pAgo comprising a polynucleotide sequence of SEQ ID NO:9 or a sequence hybridisable thereto, preferably under stringent conditions, having binding activity for a ssDNA guide, and nuclease activity for a target DNA; whereby: a first pAgo is bound to a first ssDNA guide to form a first pAgo-guide complex and a second pAgo is bound to a second ssDNA guide to form a second pAgo-guide complex; and both first and second pAgo-guide complexes are associated with a target DNA, and there is cleavage of the double stranded target DNA.

The invention will now be described in detail with reference to particular embodiments and with reference to the examples and drawings in which:

### DESCRIPTION OF THE FIGURES

FIG. 1 shows a phylogenetic tree of Ago proteins.

FIG. 2 shows a sequence alignment between different mesophilic pAgos. Clostridium bartletti (annotated: CbartAgo), Natronobacterium gregoryi (annotated: NgAgo), Synechococcus elongatus (annotated: SeAgo) and Clostridium butyricum (annotated: CbAgo). Regions shaded in grey indicate the OB-fold domain of NgAgo (annotated: OB-fold) and the PIWI domain (annotated: PIWI). The percentage amino acid identity with the amino acid sequence of CbAgo are 23% (NgAgo), 25% (SeAgo) and 36% (CbartAgo).

FIG. 3A is a sequence alignment showing the regions where the catalytic DEDX tetrad of seventeen Agos are. The tetrad is responsible for the nuclease activity of Argonaute, and the alignment shows that the core catalytic residues are also conserved in CbAgo.

FIG. 3B is a plasmid map showing the ligation independent cloning vectors used.

FIG. 3C is an overview of preparation of backbones and inserts as well as vector-insert annealing.

FIG. 3D is an SDS-PAGE gel showing His-MBP-CbAgo purification elution fraction gel analysis after the protein was purified from a HisTrap column, then a heparin column and finally using size exclusion chromatography (SEC). The expected size of CbAgo including the MBP tag is 129 kDa (calculated with http://www.expasy.org/).

FIG. 3E is an agarose gel showing that MBP-CbAgo cleavage efficiency is unaffected by a month of −80° C. storage.

FIG. 4 shows the sequences of the ssDNA and ssRNA guide tested with DNA and RNA target sequences, with arrows indicating the predicted site of cleavage.

FIG. 5 shows that Mbp-tagged CbAgo complexed with ssDNA guides leads to cleavage of ssDNA targets. No cleavage of ssRNA or ssDNA was observed when an ssRNA guide was used.

FIG. 6 shows the results of four electromobility shift assays (EMSAs) with CbAgo resolved by native polyacrylamide gel electrophoresis to test the binding capacity with the four guide/target combinations.

FIG. 7A shows a urea/polyacrylamide gel with the products from a DNA/DNA guide/target temperature activity assay. DNA guides were incubated 15 min with CbAgo and Mn²⁺ cations at room temperature before adding target. 11 nt product bands appeared between 32-44° C. indicating an efficient reaction. FIG. 7B shows a urea/polyacrylamide gel with the results of a wide-range temperature activity assay, showing a temperature range at which CbAgo is able to cleave targets around 37° C. as almost all target has been cleaved into product after one hour.

FIG. 8A shows a urea/polyacrylamide gel with the results of an activity assay at 37° C., 1 hour using 8 different cations, all supplied before guide acquisition at 250 μM. FIG. 8B shows a urea/polyacrylamide gel with the results of an activity assay with 25-1000 μM of supplied cation. Contrariwise to 8A, no cleavage is observed for Zn²⁺. FIG. 8C shows a urea/polyacrylamide gel with the results of an activity assays showing cleavage efficiency difference between Mn⁺ and Mg²⁺ at 2.5 μM. Further, Zn²⁺ was shown again to be unable to mediate cleavage. EDTA was used as a negative control because it chelates cations, resulting in a CbAgo free of cations and thus is unable to cleave.

FIG. 9 shows guide, target and products resolved on urea/polyacrylamide gel after incubation with CbAgo. Activity assays with different molar ratios CbAgo: DNA Guide: DNA Target. Equal concentrations of target were loaded on gel, explaining the absence of some guide bands. FIG. 9A shows a time-series of different molar ratios within an hour. Product bands appear quickly (Not visible for ratio D due to dilution and target excess). FIG. 9B shows all of the controls as well as reactions after 4 hours.

FIG. 10 shows a guide, target and products resolved on urea/polyacrylamide gel after incubation with CbAgo and PfAgo. The results suggest that CbAgo retains its original DNA guide even when subsequently incubated with another DNA guide.

FIG. 11 shows urea/polyacrylamide gel with the products of DNAse and RNAse cleavage assays when CbAgo was incubated for 5 min at 37° C. Only when CbAgo was combined with single stranded DNA guide, was the DNA guide protected from DNAse digestion.

FIG. 12 shows plasmid-guide alignments with the sequences of single stranded DNA guides designed to target a specific sequence on a dsDNA plasmid (pWUR704). The black arrows indicate the predicted cleavage sites.

FIG. 13 shows guide, target and products resolved on a urea/polyacrylamide gel after incubation with CbAgo. The results show that when CbAgo is incubated with both FW and RV guides, CbAgo is able to linearize a supercoiled pWUR704 plasmid. CbAgo incubated with a single DNA guide resulted in an increased proportion of open-circularised plasmid.

### DETAILED DESCRIPTION

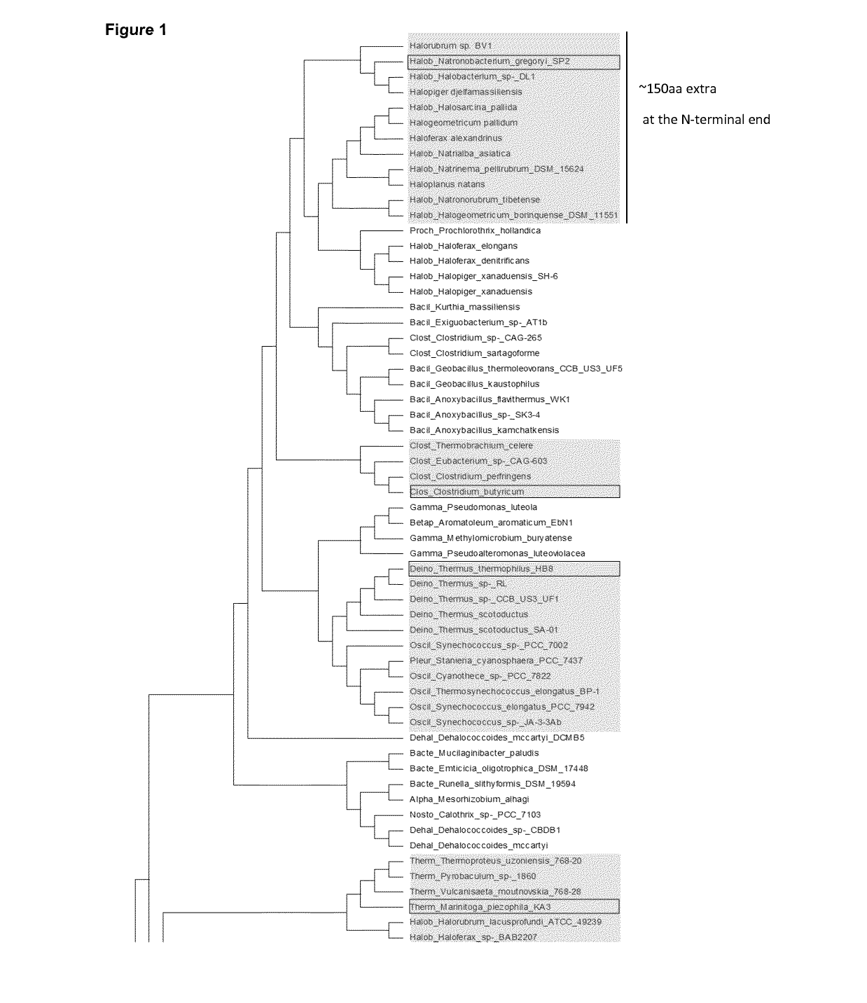

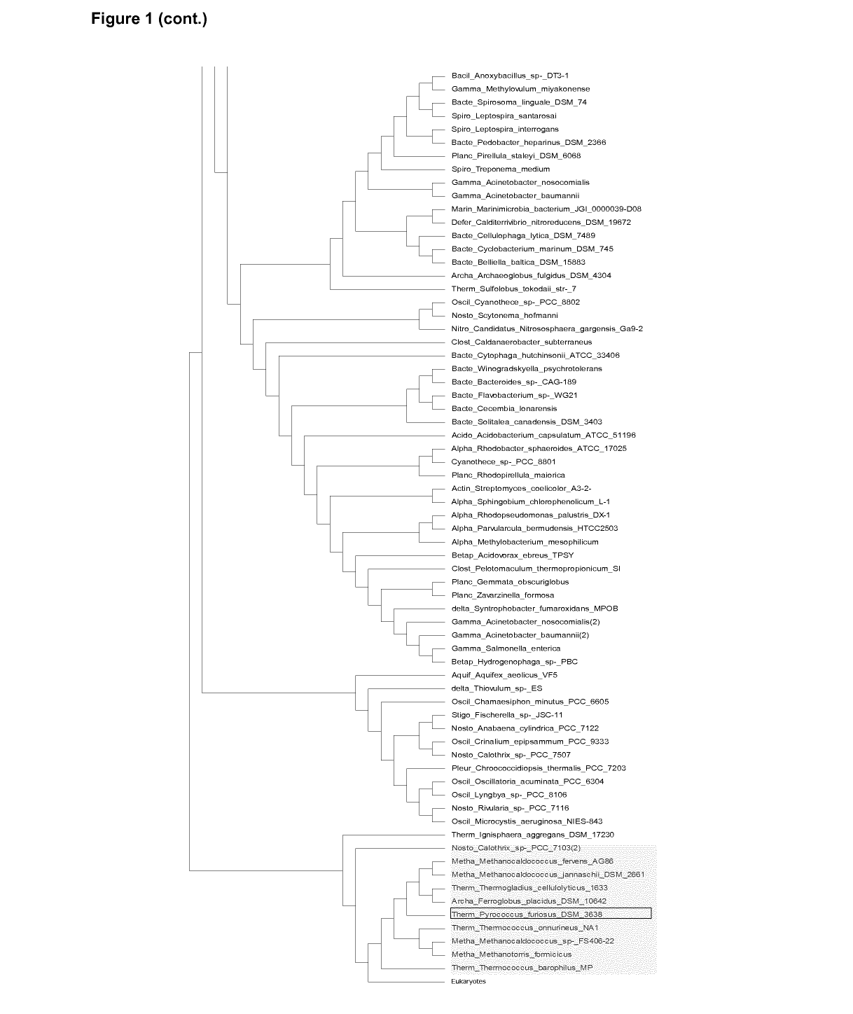

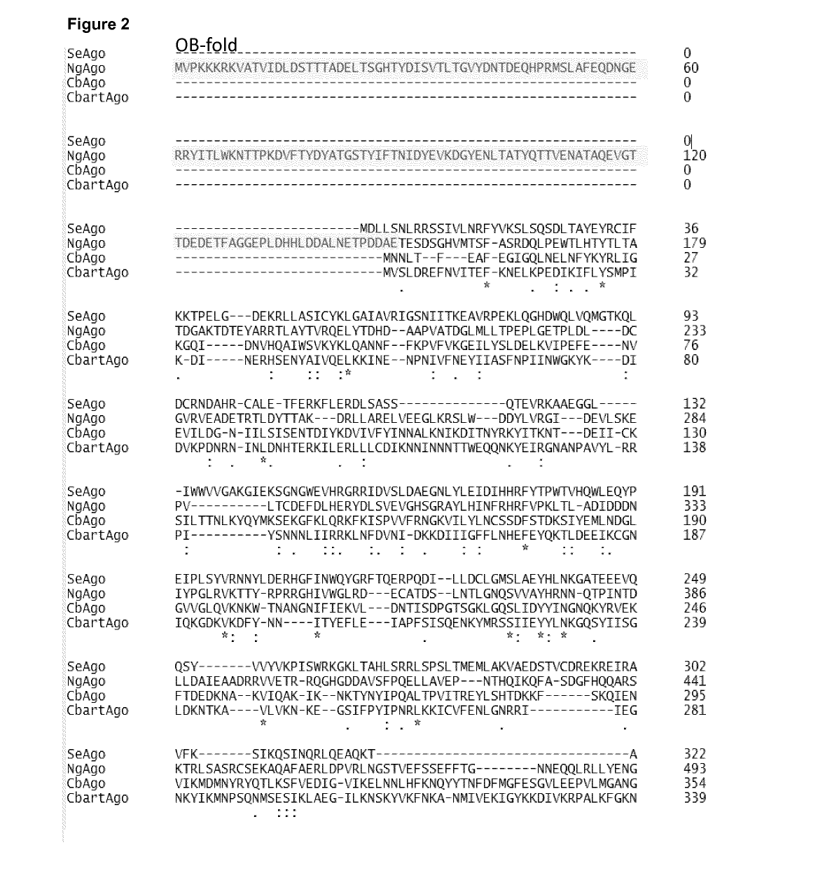

The sequences of the MID-PIWI domains of the pAgos were aligned using Muscle (using MEGA 6). From these alignments a maximum likelihood phylogenetic unrooted tree was created using MEGA 6 (FIG. 1). A particular clade of Argonautes from the Halobacteriaceae family all had approximately an additional 150 amino acids located at the N-terminus (FIGS. 1 and 2).

TABLE 1
Domain prediction pAgos based on TtAgo structure

| Domain | CbartAgo | SeAgo | CbAgo | NgAgo |
|---|---|---|---|---|
| Ob-fold | | | | 1-149 |
| N-domain | 1-84 | 1-96 | 1-94 | 150-248 |
| L1 | 85-163 | 97-182 | 95-164 | 249-315 |
| PAZ | 164-282 | 183-304 | 165-287 | 316-426 |
| L2 | 283-370 | 305-381 | 288-368 | 427-502 |
| MID | 371-513 | 382-512 | 369-527 | 503-647 |
| PIWI | 514-736 | 513-735 | 528-748 | 649-884 |

Protein BLAST data indicated that these 150 amino acids encode an OB-fold domain (for specific residue numbers (see Table 1 which was made by aligning the indicated pAgos with TtAgo in phyre2 (http://www.sbg.bio.ic.ac.uk/phyre2/html/page.cgi?id=index)). One of the known functions of the OB-fold domain is polynucleotide binding. This extra domain is lacking in CbAgo.

pAgo

In certain embodiments, a pAgo of the invention may comprise an amino acid sequence of at least 50% identity; preferably at least 80%; more preferably at least 90%; even more preferably at least 95% identity to SEQ ID NO: 1.

Where the pAgo of the invention comprises an amino acid sequence having a percentage identity with an amino acid sequence of SEQ ID NO:1 which is at least 50%, alternatively, the percentage identity may be selected from one of at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or at least 99.8%.

Regarding polynucleotide hybridisation conditions these are familiar to the skilled reader in the field. Hybridization of a nucleic acid molecule occurs when two complementary nucleic acid molecules undergo an amount of hydrogen bonding to each other known as Watson-Crick base pairing. The stringency of hybridization can vary according to the environmental (i.e. chemical/physical/biological) conditions surrounding the nucleic acids, temperature, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes

Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Allows Sequences that Share at Least 80% Identity to Hybridize)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Allows Sequences that Share at Least 50% Identity to Hybridize)

Hybridization: 6×SSC at RT to 55° C. for 16-20 hours

Wash at least twice: 2×-3×SSC at RT to 55° C. for 20-30 minutes each.

Where the pAgo of the invention comprises a PIWI domain having a percentage identity with an amino acid sequence of SEQ ID NO:3 of at least 50% then alternatively the percentage identity may be selected from one of at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or at least 99.8%.

The percentage amino acid sequence identity with SEQ ID NO: 1, 2 and/or 3 is determinable as a function of the number of identical positions shared by the sequences in a selected comparison window, taking into account the number of gaps, and the length of each gap, which need to be introduced for optimal alignment of the two sequences. Various methods of sequence identity comparison are well known to a person of average skill in the art, e.g. sequence identity may be determined by way of BLAST and subsequent Cobalt multiple sequence alignment at the National Center for Biotechnology Information webserver, where the sequence in question is compared to a reference sequence (e.g. SEQ ID NO: 1, 2 or 3). The amino acid sequences may be defined in terms of percentage sequence similarity based on a BLOSUM62 matrix or percentage identity with a given reference sequence (e.g. SEQ ID NO: 1, 2 or 3). The similarity or identity of a sequence involves an initial step of making the best alignment before calculating the percentage conservation with the reference and reflects a measure of evolutionary relationship of sequences.

A pAgo of the invention may be characterised in terms of both the reference sequence SEQ ID NO: 1 and any aforementioned percentage variant thereof as defined by percentage sequence identity, alone or in combination with any of the aforementioned amino acid motifs (i.e. SEQ ID NO: 2 and/or 3) as essential features.

Also, the invention provides polynucleotides encoding any of the aforementioned pAgos of the invention. The polynucleotides may be isolated or in the form of expression constructs, as described herein. Alternatively, the invention provides an mRNA encoding any of the aforementioned pAgos. In further aspects, the invention may provide a complementary DNA (cDNA) polynucleotide encoding a pAgo as described herein. The polynucleotide encoding a pAgo as described herein may have codon-optimisation for expression in a specific host expression cell. Additionally, the pAgos described herein may further comprise a labelling agent e.g a fluorescent label and/or a peptide/protein tag.

In all aforementioned aspects of the present invention, amino acid residues may be substituted conservatively or non-conservatively. Conservative amino acid substitutions refer to those where amino acid residues are substituted for other amino acid residues with similar chemical properties (e.g., charge or hydrophobicity) and therefore do not alter the functional properties of the resulting polypeptide.

In all aforementioned aspects of the present invention, amino acid residues may be substituted conservatively or non-conservatively. Conservative amino acid substitutions refer to those where amino acid residues are substituted for other amino acid residues with similar chemical properties (e.g., charge or hydrophobicity) and therefore do not alter the functional properties of the resulting polypeptide. Similarly it will be appreciated by the skilled reader that nucleic acid sequences may be substituted conservatively or non-conservatively without affecting the function of the polypeptide. Conservatively modified nucleic acids are those substituted for nucleic acids which encode identical or functionally identical variants of the amino acid sequences. It will be appreciated by the skilled reader that each codon in a nucleic acid (except AUG and UGG; typically the only codons for methionine or tryptophan, respectively) can be modified to yield a functionally identical molecule. Accordingly, each silent variation (i.e. synonymous codon) of a polynucleotide or polypeptide, which encodes a polypeptide of the present invention, is implicit in each described polypeptide sequence.

Similarly, it will be appreciated by a person of average skill in the art that polynucleotide sequences may be substituted conservatively or non-conservatively without affecting the function of the polypeptide. Conservatively modified polynucleotides are those substituted for nucleic acids which encode identical or functionally identical variants of the amino acid sequences. It will be appreciated by the skilled reader that each codon in a nucleic acid (except AUG and UGG; typically the only codons for methionine or tryptophan, respectively) can be modified to yield a functionally identical molecule. Accordingly, each silent variation (i.e. synonymous codon) of a polynucleotide or polypeptide, which encodes a polypeptide of the present invention, is implicit in each described polypeptide sequence.

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P, H | Other constrained (P, H) |
| N, Q, S, T | Other polar (N, Q, S, T) |
| Y, W, F | Other aromatic (Y, W, F) |
| C | None |

In some embodiments of the invention, the pAgo may be obtained or derived from bacteria, archaea or viruses; or alternatively may be synthesised de novo.

In some embodiments, a pAgo of the invention is derived from a prokaryotic organism, which may be classified as an archaea or bacterium, preferably gram positive bacterium. In some embodiments a pAgo of the invention will be derived from a mesophilic bacterium. Herein, the term mesophilic is to be understood as meaning capable of survival and growth at moderate temperatures, for example in the context of the invention, capable of polynucleotide cleavage between 10 and 50° C. In some embodiments, a pAgo of the invention may be isolated from one or more mesophilic bacteria and functions above 15° C. In some embodiments, a pAgo of the invention may be isolated from one or more mesophilic bacteria and will function in the range 20° C. to 40° C. and ideally at 37° C.

It is contemplated that in some embodiments of the invention a pAgo, may be synthesised de novo. Such de novo synthesised pAgo can comprise a non-naturally occurring fusion protein wherein the pAgo further comprises advantageous domains and/or functionality. In some embodiments, the pAgo of the present invention does not comprise an N-terminal OB (oligonucleotide/oligosaccharide-binding) fold-domain as outlined in SEQ ID NO:2, or sequence of at least 80% identity therewith. Herein, the term OB-fold domain is defined as a five/six-stranded closed beta-barrel formed by 70-80 amino acid residues. The strands are connected by loops of varying length which form the functional appendages of the protein. The majority of OB-fold domain proteins use the same face for ligand binding or as an active site. Different OB-fold domain proteins use this ‘fold-related binding face’ to, variously, bind oligosaccharides, oligonucleotides, proteins, metal ions and catalytic substrates. Many OB-fold domains bind to polynucleotides. The OB-fold domain is found in all three kingdoms and its common architecture presents a binding face that has adapted to bind different ligands.

More particularly, an OB-fold domain comprises an amino acid sequence with a percentage identity with SEQ ID NO:2 as follows: at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or at least 99.8%.

### Oligonucleotide Guide Design

An oligonucleotide guide for loading into a pAgo in accordance with the invention can be phosphorylated. Advantageously, the guide may be either a 5′ phosphorylated single stranded DNA or 5′ phosphorylated single stranded RNA. In some embodiments, the guide sequence comprises further phosphorylation at the 5′ end of the guide oligonucleotide such as a terminal 5′-diphosphate or preferably a terminal 5′-triphosphate.

In preferred embodiments, the polynucleotide guide is not displaceable within a pAgo-guide complex by a subsequently provided polynucleotide guide. Consequently, guide binding occurs in a one-guide faithful manner. It is contemplated that in some embodiments the one-guide faithful binding is dependent upon the temperature at which the pAgo-guide complex is maintained. In some embodiments, the temperature is in the range of 10-50° C. 15-50° C., optionally 20-50° C., 25-50° C., 30-50° C., 35-50° C., 40-50° C., 45-50° C., or any range derivable therein. Preferably, the temperature range is 25-45° C., 26-45° C., 27-45° C. 28-45° C., 29-45° C., 30-45° C., 31-45° C., 32-45° C., 32-44° C., 33-45° C., 34-45° C., 35-45° C., 36-45° C., 37-45° C., 33-45° C., 39-45° C. or any range derivable therein. More preferably the temperature is 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C. or 44° C.

An oligonucleotide guide for loading into a pAgo may be in the range of 10 to 50 nucleotides or any range derivable therein, optionally 15 to 50 nucleotides, 20 to 50 nucleotides, 25 to 50 nucleotides, 30 to 50 nucleotides, 35 to 50 nucleotides, 40 to 50 nucleotides, 45 to 50 nucleotides in length, preferably 15 to 30 nucleotides in length, more preferably 20 to 25 nucleotides in length.

An oligonucleotide guide for loading into a pAgo in accordance with the invention may be at least 10 nucleotides, generally at least about 13 nucleotides, e.g. 13, 14 or 15 nucleotides, but typically not more than about 50 nucleotides, more preferably not more than 25, 24, 23, 22, 21, 20, 19, 18, 17 or 16 nucleotides in length. Conveniently, for example, a 21 nucleotide oligonucleotide guide may be employed.

Typically, the portion of the oligonucleotide guide which is fully complementary to the target strand will extend from at least nucleotide 2, 3 or 4 to nucleotide 16 (determined with reference from 5′-end of the guide) so as to facilitate efficient target cleavage, although a shorter stretch of complementary sequence may suffice, e.g. nucleotides 2 to 8 in a far shorter oligonucleotide guide, e.g. of 9 nucleotides, which does not provide a 3′-end which contacts a PAZ pocket. The oligonucleotide guides may be conveniently chosen to be fully complementary to the target. Without wishing to be bound to particular mechanism, the inventors expected that the oligonucleotide guide will direct cleavage of its target strand at the bond between the 10th and 11th nucleotides (t10 and t11) determined with reference to the 5′-end of the guide.

The precise design for optimum cleavage of the selected target cleavage site may be determined by preliminary tests with plasmid targets incorporating the target site.

Advantageously, oligonucleotide guides may be combined with pAgos of the invention at room temperature or assay temperature. Once incubated together there is formation of pAgo-guide complexes that can efficiently target and site-specifically cleave or bind target DNA or bind target RNA. Consequently, for in vitro applications, it is not necessary to pre-heat the pAgos and guides at elevated temperatures in order to create a pAgo-guide complex suitable for cleaving or binding a target DNA or binding a target RNA.

A further advantage is that recombinant pAgos, of the invention, expressed in, for example an E. coli, host cells do not sequester polynucleotides that are produced by host cells. Such incorporation has been observed for other mesophilic pAgos. The polynucleotides that are incorporated non-specifically into pAgos could result in off-target cleavage within the target DNA. Consequently, with other mesophilic pAgos a step is required to replace the non-specifically bound polynucleotides from pAgos, which typically use elevated temperatures that could impact enzyme activity. In contrast, when recombinant pAgo of the present invention is purified it is already in a suitable state to be loaded with a suitable ssDNA or ssRNA guide.

Where a pair of pAgo-DNA guide complexes are provided for targeting complementary strands of a double stranded DNA, as indicated above, preferably the pair of guides may target overlapping or partially overlapping complementary sequences, so as to generate either blunt or staggered (also referred to as overhangs or sticky ends) cleavage products. In order to target overlapping or partially overlapping complementary sequences, the first ssDNA guide and second ssDNA guide are preferably substantially complementary in sequence.

### Oligonucleotide Targets

As described above, the invention provides a pAgo and a DNA guide, which form a complex so as to cleave a target DNA.

An advantageous aspect of the present invention is that the pAgo can target both single stranded DNA and double stranded DNA.

In some embodiments, the double stranded target DNAs are plasmids, preferably a negatively supercoiled plasmid. In certain embodiments the plasmid is already nicked. In further embodiments, the target DNA is a linear fragment, including a linearised plasmid. It is also contemplated that in some embodiments the target DNA is genomic or chromosomal.

In all aforementioned embodiments of the invention the target DNA is either nicked or cleaved. As used herein, a nick is a discontinuity in a double stranded polynucleotide in which there is no phosphodiester bond between adjacent nucleotides of one strand of the polynucleotide.

Whilst other mesophilic pAgos are known to cleave site-specifically when bound to an ssDNA guide, they have also been observed to remove several nucleotides at random (Gao et al. 2016 ibid.). This feature limits the ability of such enzymes to be used for genome editing and other applications in molecular biology requiring a high level of precision.

Advantageously, a pAgo of the present invention, cleaves site specifically when bound to an ssDNA guide but has not been observed to remove any nucleotides at random within the target DNA. This allows pAgos to be used for precise applications. For example, pAgos could be used to generate blunt or staggered cuts in the target DNA. As used herein, cleavage results in a double stranded DNA in which there is no phosphodiester bond between adjacent nucleotides on both strands of the polynucleotide. In some embodiments, the resulting cleavage product is referred to as being blunt-ended. As used herein, a blunt-ended cleavage product results when both strands are cut at a single base pair, so as to produce no overhangs. Blunt-ended products contrast with a product with a staggered end. These products are not blunt but instead have additional nucleotides on one polynucleotide strand. These overhangs can be any number of nucleotides in length and located on either strand of the double stranded polynucleotide.

Alternatively, a pAgo as described herein and an ssDNA or ssRNA guide can form a complex so as to site-specifically bind (but not cleave) a target RNA.

### Cations

Advantageously, a pAgo of the present invention has site-specific cleavage activity in the presence of a number of different divalent cations. Specifically, cleavage is observed in the presence of Mn⁺⁺, Mg⁺⁺, Ca⁺⁺, Cu⁺⁺, Fe⁺⁺, Co⁺⁺, Zn⁺⁺, Ni⁺⁺. Preferably, the divalent cation used by a pAgo is Mn⁺⁺.

A range of concentrations are suitable for site-specific cleavage by a pAgo. In some embodiments, the concentration is in the range of 1-100 μM, 100-200 μM, 200-300 μM, 300-400 μM, 400-500 μM, 500-600 μM, 600-700 μM, 700-800 μM, 900-1000 μM, or any range derivable therein. Preferably, the concentration is in the range of 1-10 μM, 10-20 μM, 20-30 μM, 30-40 μM, 40-50 μM, 50-60 μM, 60-70 μM, 70-80 μM, 90-100 μM, 100-110 μM, 110-120 μM, 120-130 μM, 130-140 μM, 140-150 μM, 150-160 μM, 160-170 μM, 170-180 μM, 190-200 μM, 200-210 μM, 210-220 μM, 220-230 μM, 230-240 μM, 240-250 μM, or any range derivable therein.

### Compositions

Another aspect of the present invention provides a composition comprising at least one pAgo as described herein, and at least one single stranded polynucleotide guide as described herein. It is contemplated that any of the pAgos described herein can be combined in a composition with any single stranded guide polynucleotide described herein. Optionally, in some embodiments a composition further comprises a target polynucleotide that comprises a nucleotide sequence that is substantially complementary to at least one single stranded polynucleotide guide.

### Cleavage Temperatures

In another aspect, the present invention provides an isolated pAgo protein or polypeptide fragment thereof having an amino acid sequence of SEQ ID NO: 1 or a sequence of at least 50% identity therewith, wherein the pAgo protein or polypeptide is capable of cleavage or nicking of a target DNA at a temperature in the range 15° C. and 60° C. inclusive.

Preferably, pAgo proteins or polypeptides of the invention, when associated with suitable ssDNA guide which recognizes a sequence within a target DNA molecule(s) to be cleaved, nicked or modified, does so at temperatures in the range 10° C. to 50° C., optionally in the range 10° C. to 50° C., 15° C. to 50° C., 20° C. to 50° C., 25° C. to 50° C., 30° C. to 50° C., 35° C. to 50° C., 40° C. to 50° C. or 45° C. to 50° C., or any range derivable therein.

Preferably, the pAgo protein or polypeptide is, when associated with suitable ssDNA guide which recognizes a sequence in the target DNA molecule(s) to be cleaved, nicked or modified, does so at temperatures in the range 32° C. to 44° C. inclusive. For example, the cleavage, nicking or modifying occurs at a temperature of 32° C., 33° C., 34° C., 35° C., 36° C., 37° C., 38° C., 39° C., 40° C., 41° C., 42° C., 43° C. or 44° C. More preferably the pAgo protein or polypeptide is capable of cleaving, nicking or marking at a temperature of 37° C.

### Expression Vectors

Polynucleotides of the present invention may be isolated. However, in order that expression of the polynucleotide construct may be carried out in a chosen cell, the polynucleotide sequence encoding the pAgo protein will preferably be provided in an expression construct. In some embodiments, the polynucleotide encoding the pAgo will be provided as part of a suitable expression vector. In some embodiments, the expression vector is a virus or viral vector. In other embodiments the viral or virus expression vector is retrovirus or lentivirus vectors. An ssDNA guide as hereinbefore defined may be delivered to a target cell by other means. Consequently, such expression vectors and ssDNA guide can be used in an appropriate host to generate a pAgo-guide complex of the invention which can target a desired target DNA.

Suitable expression vectors will vary according to the recipient cell and suitably may incorporate regulatory elements which enable expression in the target cell and preferably which facilitate high-levels of expression. Such regulatory sequences may be capable of influencing transcription or translation of a gene or gene product, for example in terms of initiation, accuracy, rate, stability, downstream processing and mobility.

Such elements may include, for example, strong and/or constitutive promoters, 5′ and 3′ UTR's, transcriptional and/or translational enhancers, transcription factor or protein binding sequences, start sites and termination sequences, ribosome binding sites, recombination sites, polyadenylation sequences, sense or antisense sequences, sequences ensuring correct initiation of transcription and optionally poly-A signals ensuring termination of transcription and transcript stabilisation in the host cell. The regulatory sequences may be plant-, animal-. bacterial-, fungal- or virus derived, and preferably may be derived from the same organism as the host cell. Clearly, appropriate regulatory elements will vary according to the host cell of interest. For example, regulatory elements which facilitate high-level expression in prokaryotic host cells such as in E. coli may include the pLac, T7, P(Bla), P(Cat), P(Kat), trp or tac promoters. Regulatory elements which facilitate high-level expression in eukaryotic host cells might include the AOX1 or GAL1 promoter in yeast or the CMV- or SV40-promoters, CMV-enhancer, SV40-enhancer, Herpes simplex virus VIP16 transcriptional activator or inclusion of a globin intron in animal cells. In plants, constitutive high-level expression may be obtained using, for example, the Zea mays ubiquitin 1 promoter or 35S and 19S promoters of Cauliflower mosaic virus (CaMV).

Suitable regulatory elements may be constitutive, whereby they direct expression under most environmental conditions or developmental stages, developmental stage specific or inducible. Preferably, the promoter is inducible, to direct expression in response to environmental, chemical or developmental cues, such as temperature, light, chemicals, drought, and other stimuli. Suitably, promoters may be chosen which allow expression of the protein of interest at particular developmental stages or in response to extra- or intra-cellular conditions, signals or externally applied stimuli. For example, a range of promoters exist for use in E. coli which give high-level expression at particular stages of growth (e.g. osmY stationary phase promoter) or in response to particular stimuli (e.g. HtpG Heat Shock Promoter).

Suitable expression vectors may comprise additional sequences encoding selectable markers which allow for the selection of said vector in a suitable host cell and/or under particular conditions.

Suitable expression vectors may further comprise additional sequences which allow for the localisation of the pAgo to a specific organelle in a eukaryotic cell. For example, an expression vector encoding a pAgo could further comprise a nuclear localisation sequence (NLS) at either C-terminus, N-terminus or at both termini of the pAgo so that once expressed in a eukaryotic cell, the pAgo would be localised to the nucleus. A number of different NLS are known in the art (Nair et al., Nucleic Acids Research, (2003)). It is also contemplated that an expression sequence may also include localisation sequences targeting pAgos to the mitochondria or chloroplasts of a eukaryotic cell.

### Methods

The invention also includes a method of modifying a target DNA in a cell, comprising the steps of transfecting, transforming or transducing the cell with any of the expression vectors as hereinbefore described. The methods of transfection, transformation or transduction are of the types well known to a person of skill in the art. Where there is one expression vector used to generate expression of a pAgo of the invention and when the ssDNA guide is added directly to the cell then the same or a different method of transfection, transformation or transduction may be used. Similarly, when there is one expression vector being used to generate expression of a pAgo of the invention and when another expression vector is being used to generate expression of a second pAgo, then the same or a different method of transfection, transformation or transduction may be used.

In other embodiments, mRNA encoding the pAgo protein or polypeptide is introduced into a cell so that the complex is expressed in the cell. The ssDNA guide which guides the pAgo protein complex to the appropriate target DNA is also introduced into the cell, whether simultaneously, separately or sequentially from the mRNA, such that the necessary pAgo-guide complex is formed in the cell.

Accordingly, the invention also provides a method of modifying, i.e. cleaving, tagging, marking or binding, a target DNA comprising the step of contacting the polynucleotide with a pAgo-guide complex as hereinbefore defined.

In accordance with the above methods, modification of a target DNA may therefore be carried out in vitro and in a cell-free environment. In a cell-free environment, addition of each of the target DNA, the pAgo protein and the ssDNA guide may be simultaneous, sequential (in any order as desired), or separately. Thus it is possible for the target DNA and ssDNA guide to be added simultaneously to a reaction mix and then the pAgo protein or polypeptide of the invention to be added separately at a later stage.

Equally, the modification of the target DNA may be made in vivo, that is in situ in a cell, whether an isolated cell or as part of a multicellular tissue, organ or organism. In the context of whole tissue and organs, and in the context of an organism, the method may desirably be carried out in vivo or alternatively may be carried out by isolating a cell from the whole tissue, organ or organism, treating the cell pAgo-guide complex in accordance with the method and subsequently returning the cell treated with pAgo-guide complex to its former location, or a different location, whether within the same or a different organism.

In these embodiments, the pAgo-guide complex or the pAgo protein or polypeptide requires an appropriate form of delivery into the cell. Such suitable delivery systems and methods are well known to persons skilled in the art, and include but are not limited to cytoplasmic or nuclear microinjection. In preferred modes of delivery, an Adeno-associated virus (AAV) is used; this delivery system is not disease causing in humans and has been approved for clinical use in Europe.

### Cells

Advantageously, the present invention is of broad applicability and cells of the present invention may be derived from any genetically tractable organism which can be cultured. Accordingly, the present invention provides a cell transformed by a method as hereinbefore described.

Appropriate cells may be prokaryotic or eukaryotic. In particular, commonly used cells may be selected for use in accordance with the present invention including prokaryotic or eukaryotic cells which are genetically accessible and which can be cultured, for example prokaryotic cells, fungal cells, plant cells and animal cells including human cells (but not embryonic stem cells). Preferably, cells will be selected from a prokaryotic cell or a eukaryotic cell. Preferred cells for use in accordance with the present invention are commonly derived from species which typically exhibit high growth rates, are easily cultured and/or transformed, display short generation times, species which have established genetic resources associated with them or species which have been selected, modified or synthesized for optimal expression of heterologous protein under specific conditions. In preferred embodiments of the invention where the pAgo of interest is eventually to be used in specific industrial, agricultural, chemical or therapeutic contexts, an appropriate cell may be selected based on the desired specific conditions or cellular context in which the protein of interest is to be deployed. Preferably the cell will be a prokaryotic cell. In preferred embodiments the cell is a bacterial cell. The cell may for instance be an Escherichia coli (E. coli) cell.

[SEQ ID NO: 1]
Amino acid sequence of Clostridium butyricum (CbAgo)
MNNLTFEAFEGIGQLNELNFYKYRLIGKGQIDNVHQAIWSVKYKLQANNFFKPVFVKGEILYSLDELKVI
PEFENVEVILDGNIILSISENTDIYKDVIVFYINNALKNIKDITNYRKYITKNTDEIICKSILTTNLKYQ
YMKSEKGFKLQRKFKISPVVFRNGKVILYLNCSSDFSTDKSIYEMLNDGLGVVGLQVKNKWTNANGNIFI
EKVLDNTISDPGTSGKLGQSLIDYYINGNQKYRVEKFTDEDKNAKVIQAKIKNKTYNYIPQALTPVITRE
YLSHTDKKFSKQIENVIKMDMNYRYQTLKSFVEDIGVIKELNNLHFKNQYYTNFDFMGFESGVLEEPVLM
GANGKIKDKKQIFINGFFKNPKENVKFGVLYPEGCMENAQSIARSILDFATAGKYNKQENKYISKNLMNI
GFKPSECIFESYKLGDITEYKATARKLKEHEKVGFVIAVIPDMNELEVENPYNPFKKVWAKLNIPSQMIT
LKTTEKFKNIVDKSGLYYLHNIALNILGKIGGIPWIIKDMPGNID [symbol] EKGIHFPACSVLFDK
YGKLINYYKP [symbol] ILQEIFDNVLISYKEENGEYPKN [symbol] NIDWYKEYFDKKGI
KFNIIEVKKNIPVKIAKVVGSNICNPIKGSYVLKNDKAFIVTTDIKDGVASPNPLKIEKTYGDVEMKSIL
EQIYSLSQIHVGSTKSLRLPI [symbol] IEYIPQGVVDNRLFFL
[SEQ ID NO: 2]
Amino acid sequence of an OB-fold domain
MTVIDLDSTTTADELTSGHTYDISVTLTGVYDNTDEQHPRMSLAFEQDNGERRYITLWKNTTPKDVFTYD
YATGSTYIFTNIDYEVKDGYENLTATYQTTVENATAQEVGTTDEDETFAGGEPLDHHLDDALNETPDDAE
TESDSGHVM
[SEQ ID NO: 3]
Amino acid sequence of the CbAgo PIWI domain
KDMPGNIDCFIGLDVGTREKGIHFPACSVLFDKYGKLINYYKPTIPQSGEKIAETILQEIFDNVLISYKE
ENGEYPKNIVIHRDGFSRENIDWYKEYFDKKGIKFNIIEVKKNIPVKIAKVVGSNICNPIKGSYVLKNDK
AFIVTTDIKDGVASPNPLKIEKTYGDVEMKSILEQIYSLSQIHVGSTKSLRLPITTGYADKICKAIEYIP
QGVVDNRLFFL
[SEQ ID NO: 4]
Amino acid sequence of Natronobacterium gregoryi (NgAgo)
MVPKKKRKVATVIDLDSTTTADELTSGHTYDISVTLTGVYDNTDEQHPRMSLAFEQDNGERRYITLWKNT
TPKDVFTYDYATGSTYIFTNIDYEVKDGYENLTATYQTTVENATAQEVGTTDEDETFAGGEPLDHHLDDA
LNETPDDAETESDSGHVMTSFASRDQLPEWTLHTYTLTATDGAKTDTEYARRTLAYTVRQELYTDHDAAP
VATDGLMLLTPEPLGETPLDLDCGVRVEADETRTLDYTTAKDRLLARELVEEGLKRSLWDDYLVRGIDEV
LSKEPVLTCDEFDLHERYDLSVEVGHSGRAYLHINFRHRFVPKLTLADIDDDNIYPGLRVKTTYRPRRGH
IVWGLRDECATDSLNTLGNQSVVAYHRNNQTPINTDLLDAIEAADRRVVETRRQGHGDDAVSFPQELLAV
EPNTHQIKQFASDGFHQQARSKTRLSASRCSEKAQAFAERLDPVRLNGSTVEFSSEFFTGNNEQQLRLLY
ENGESVLTFRDGARGAHPDETFSKGIVNPPESFEVAVVLPEQQADTCKAQWDTMADLLNQAGAPPTRSET
VQYDAFSSPESISLNVAGAIDPSEVDAAFVVLPPDQEGFADLASPTETYDELKKALANMGIYSQMAYFDR
FRDAKIFYTRNVALGLLAAAGGVAFTTEHAMPGDADMFIGIDVSRSYPEDGASGQINIAATATAVYKDGT
ILGHSSTRPQLGEKLQSTDVRDIMKNAILGYQQVTGESPTHIVIHRDGFMNEDLDPATEFLNEQGVEYDI
VEIRKQPQTRLLAVSDVQYDTPVKSIAAINQNEPRATVATFGAPEYLATRDGGGLPRPIQIERVAGETDI
ETLTRQVYLLSQSHIQVHNSTARLPITTAYADQASTHATKGYLVQTGAFESNVGFL
[SEQ ID NO: 5]
Amino acid sequence of Clostridium bartletti (CbartAgo)
MVSLDREFNVITEFKNELKPEDIKIFLYSMPIKDINERHSENYAIVQELKKINENPNIVFNEYIIASFNP
IINWGKYKDIDVKPDNRNINLDNHTERKILERLLLCDIKNNINNNTTWEQQNKYEIRGNANPAVYLRRPI
YSNNNLIIRRKLNFDVNIDKKDIIIGFFLNHEFEYQKTLDEEIKCGNIQKGDKVKDFYNNITYEFLEIAP
FSISQENKYMRSSIIEYYLNKGQSYIISGLDKNTKAVLVKNKEGSIFPYIPNRLKKICVFENLGNRRIIE
GNKYIKMNPSQNMSESIKLAEGILKNSKYVKFNKANMIVEKIGYKKDIVKRPALKFGKNESNFSAMYGLN
KSGSYEQKNIKIDYFIDPKILNNKRDYQIVYSFLNDIISKSKDLGVEINTDKSYINLTPINIKNENVFEL
NIIQIIENYNNPVLVILEKENIDKYYETLKKIFGGRNNIPTQFVDLDTIKKCDPKIDNKRGKESIFLNIL
LGIYCKSGIQPWVLANGLSADCYIGLDVCRENNMSTAGLIQVIGKDGRVLKSKTISSHQSGEKIQINILK
DIIFEAKQAYKNTYNKKLEHIVFHRDGINREDIDLLKEITNSLEIKFDYVEVTKNINRRMAMLEKSDENY
NHRDKENKKWITEIGMCLKKENEAYLITTNPSENMGMARPLRIKKVYGNQNMDDIVKDIYKLSFMHIGSI
MKSRLPITTHYADLSSIYSHRELMPKSVDNNILHFI
[SEQ ID NO: 6]
Amino acid sequence of Synechococcus elongatus (SeAgo)
MDLLSNLRRSSIVLNRFYVKSLSQSDLTAYEYRCIFKKTPELGDEKRLLASICYKLGAIAVRIGSNIITK
EAVRPEKLQGHDWQLVQMGTKQLDCRNDAHRCALETFERKFLERDLSASSQTEVRKAAEGGLIWWVVGAK
GIEKSGNGWEVHRGRRIDVSLDAEGNLYLEIDIHHRFYTPWTVHQWLEQYPEIPLSYVRNNYLDERHGFI
NWQYGRFTQERPQDILLDCLGMSLAEYHLNKGATEEEVQQSYVVYVKPISWRKGKLTAHLSRRLSPSLTM
EMLAKVAEDSTVCDREKREIRAVFKSIKQSINQRLQEAQKTASWILTKTYGISSPAIALSCDGYLLPAAK
LLAANKQPVSKTADIRNKGCAKIGETSFGYLNLYNNQLQYPLEVHKCLLEIANKNNLQLSLDQRRVLSDY
PQDDLDQQMFWQTWSSQGIKTVLVVMPWDSHHDKQKIRIQATQAGIATQFMVPLPKADKYKALNVTLGLL
CKAGWQPIQLESVDHPEVADLIIGFDTGTNRELYYGTSAFAVLADGQSLGWELPAVQGGETFSGQAIWQT
VSKLIIKFYQICQRYPQKLLLMRDGLVQEGEFQQTIELLKERKIAVDVISVRKSGAGRMGQEIYENGQLV
YRDAAIGSVILQPAERSFIMVTSQPVSKTIGSIRPLRIVHEYGSTDLELLALQTYHLTQLHPASGFRSCR
LPWVLHLADRSSKEFQRIGQISVLQNISRDKLIAV
[SEQ ID NO: 7]
DNA sequence of pWUR704
GTCGACTTTATATTTAAATAATTTAATATACTATACAACCTACTACCTCGTATAAATTTTTAAATAAATATTGCA
TTCAAGCTTTTAATTTAATTAAATGGCCGCTCTAGAGGCATCAAATAAAACGAAAGGCTCAGTCGAAAGACTGGG
CCTTTCGTTTTATCTGTTGTTTGTCGGTGAACGCTCTCCTGAGTAGGACAAATCCGCCGCCCTAGACCTAGGGTA
CGGGTTTTGCTGCCCGCAAACGGGCTGTTCTGGTGTTGCTAGTTTGTTATCAGAATCGCAGATCCGGCTTCAGGT
TTGCCGGCTGAAAGCGCTATTTCTTCCAGAATTGCCATGATTTTTTCCCCACGGGAGGCGTCACTGGCTCCCGTG
TTGTCGGCAGCTTTGATTCGATAAGCAGCATCGCCTGTTTCAGGCTGTCTATGTGTGACTGTTGAGCTGTAACAA
GTTGTCTCAGGTGTTCAATTTCATGTTCTAGTTGCTTTGTTTTACTGGTTTCACCTGTTCTATTAGGTGTTACAT
GCTGTTCATCTGTTACATTGTCGATCTGTTCATGGTGAACAGCTTTAAATGCACCAAAAACTCGTAAAAGCTCTG
ATGTATCTATCTTTTTTACACCGTTTTCATCTGTGCATATGGACAGTTTTCCCTTTGATATCTAACGGTGAACAG
TTGTTCTACTTTTGTTTGTTAGTCTTGATGCTTCACTGATAGATACAAGAGCCATAAGAACCTCAGATCCTTCCG
TATTTAGCCAGTATGTTCTCTAGTGTGGTTCGTTGTTTTTGCGTGAGCCATGAGAACGAACCATTGAGATCATGC
TTACTTTGCATGTCACTCAAAAATTTTGCCTCAAAACTGGTGAGCTGAATTTTTGCAGTTAAAGCATCGTGTAGT
GTTTTTCTTAGTCCGTTACGTAGGTAGGAATCTGATGTAATGGTTGTTGGTATTTTGTCACCATTCATTTTTATC
TGGTTGTTCTCAAGTTCGGTTACGAGATCCATTTGTCTATCTAGTTCAACTTGGAAAATCAACGTATCAGTCGGG
CGGCCTCGCTTATCAACCACCAATTTCATATTGCTGTAAGTGTTTAAATCTTTACTTATTGGTTTCAAAACCCAT
TGGTTAAGCCTTTTAAACTCATGGTAGTTATTTTCAAGCATTAACATGAACTTAAATTCATCAAGGCTAATCTCT
ATATTTGCCTTGTGAGTTTTCTTTTGTGTTAGTTCTTTTAATAACCACTCATAAATCCTCATAGAGTATTTGTTT
TCAAAAGACTTAACATGTTCCAGATTATATTTTATGAATTTTTTTAACTGGAAAAGATAAGGCAATATCTCTTCA
CTAAAAACTAATTCTAATTTTTCGCTTGAGAACTTGGCATAGTTTGTCCACTGGAAAATCTCAAAGCCTTTAACC
AAAGGATTCCTGATTTCCACAGTTCTCGTCATCAGCTCTCTGGTTGCTTTAGCTAATACACCATAAGCATTTTCC
CTACTGATGTTCATCATCTGAGCGTATTGGTTATAAGTGAACGATACCGTCCGTTCTTTCCTTGTAGGGTTTTCA
ATCGTGGGGTTGAGTAGTGCCACACAGCATAAAATTAGCTTGGTTTCATGCTCCGTTAAGTCATAGCGACTAATC
GCTAGTTCATTTGCTTTGAAAACAACTAATTCAGACATACATCTCAATTGGTCTAGGTGATTTTAATCACTATAC
CAATTGAGATGGGCTAGTCAATGATAATTACTAGTCCTTTTCCCGGGAGATCTGGGTATCTGTAAATTCTGCTAG
ACCTTTGCTGGAAAACTTGTAAATTCTGCTAGACCCTCTGTAAATTCCGCTAGACCTTTGTGTGTTTTTTTTGTT
TATATTCAAGTGGTTATAATTTATAGAATAAAGAAAGAATAAAAAAAGATAAAAAGAATAGATCCCAGCCCTGTG
TATAACTCACTACTTTAGTCAGTTCCGCAGTATTACAAAAGGATGTCGCAAACGCTGTTTGCTCCTCTACAAAAC
AGACCTTAAAACCCTAAAGGCTTAAGTAGCACCCTCGCAAGCTCGGGCAAATCGCTGAATATTCCTTTTGTCTCC
GACCATCAGGCACCTGAGTCGCTGTCTTTTTCGTGACATTCAGTTCGCTGCGCTCACGGCTCTGGCAGTGAATGG
GGGTAAATGGCACTACAGGCGCCTTTTATGGATTCATGCAAGGAAACTACCCATAATACAAGAAAAGCCCGTCAC
GGGCTTCTCAGGGCGTTTTATGGCGGGTCTGCTATGTGGTGCTATCTGACTTTTTGCTGTTCAGCAGTTCCTGCC
CTCTGATTTTCCAGTCTGACCACTTCGGATTATCCCGTGACAGGTCATTCAGACTGGCTAATGCACCCAGTAAGG
CAGCGGTATCATCAACAGGCTTACCCGTCTTACTGTCCCTAGTGCTTGGATTCTCACCAATAAAAAACGCCCGGC
GGCAACCGAGCGTTCTGAACAAATCCAGATGGAGTTCTGAGGTCATTACTGGATCTATCAACAGGAGTCCAAGCG
AGCTCTCCGTGTCGTTCTGTCCACTCCTGAATCCCATTCCAGAAATTCTCTAGCGATTCCAGAAGTTTCTCAGAG
TCGGAAAGTTGACCAGACATTACGAACTGGCACAGATGGTCATAACCTGAAGGAAGATCTCTATTCCTTTGCCCT
CGGACGAGTGCTGGGGCGTCGGTTTCCACTATCGGCGAGTACTTCTACACAGCCATCGGTCCAGACGGCCGCGCT
TCTGCGGGCGATTTGTGTACGCCCGACAGTCCCGGCTCCGGATCGGACGATTGCGTCGCATCGACCCTGCGCCCA
AGCTGCATCATCGAAATTGCCGTCAACCAAGCTCTGATAGAGTTGGTCAAGACCAATGCGGAGCATATACGCCCG
GAGCCGCGGCGATCCTGCAAGCTCCGGATGCCTCCGCTCGAAGTAGCGCGCCTGCTGCTCCATACAAGCCAACCA
CGGCCTCCAGAAGAAGATGTTGGCGACCTCGTATAGGGGATCTCCGAACATCGCCTCGCTCCAGTCAATGACCGC
TGTTATGCGGCCATTGTCCGTCAGGACATTGTTGGAGCCGAAATCCGCGTGCACGAGGTGCCGGACTTCGGGGCA
GTCCTCGGCCCAAAGCATCAGCTCATCGAGAGCCTGCGCGACGGACGCACTGACGGTGTCGTCCATCACAGTTTG
CCAGTGATACACATGGGGATCAGCAATCGCGCAGATGAAATCACGCCATGTAGTGTATTGACCGATTCCTTGCGG
TCCGAATGGGCCGAACCCGCTCGTCTGGCTAAGATCGGCCGCAGCGATCGCATCCATAACCTCCGCGACCGGTTG
CAGAACAGCGGGCAGTTCGGTTTCAGGCAGGTCTTGCAACGTGACACCCTGTGCACGGCGGGAGATGCAATAGGT
CAGGCTCTCGCTAAATTCCCCAATGTCAAGCACTTCCGGAATCGGGAGCGCGGCCGATGCAAAGTGCCGATAAAC
ATAACGATCTTTGTAGAAACCATCGGCGCAGCTATTTACCCGCAGGACATATCCACGCCCTCCTACATCGAAGCT
GAAAGCACGAGATTCTTCGCCCTCCGAGAGCTGCATCAGGTCGGAGACGCTGCCGAACTTTTCGATCAGAAACTT
CTCAACAGACGTCGCGGTGAGTTCAGGCTTTTTCATGTGCCTCACACCTCCTTAAGGGTCGTGGGCGGGAACCCG
AGACGGGCGAGTTGCCGCGTTTCCTCTCCGCCCAGGTCCGCCCGGTGCGGGGAAAACCCCCCAAAAGGAGCCCTT
TTTCCCCGCATCCGGCGCTATCGTAAAAACCTCACGCGCCCTTGTCAAACGGTCGGGCCTTAAGGTTTCTGTTAT
ACTCCCCCCGGGGATCGATCCCCGGCCCGACGGGAGCCGGGCGGTGGTGGCCTGGGCTAGC
[SEQ ID NO: 8]
DNA sequence of codon optimised CbAgo with LIC flanks
TACTTCCAATCCAATgcaAATAATCTGACCTTTGAGGCTTTTGAAGGGATTGGTCAACTGAATGAACTGAATTTT
TATAAGTATCGTCTGATTGGGAAAGGGCAGATTGATAATGTGCATCAAGCTATTTGGTCTGTTAAATATAAACTC
CAGGCTAATAATTTTTTTAAACCGGTGTTTGTGAAAGGTGAGATTCTATATAGCCTAGATGAACTGAAGGTGATT
CCGGAATTTGAAAATGTTGAGGTGATTCTCGATGGTAATATTATTCTGTCTATTTCTGAGAATACCGATATTTAT
AAGGATGTGATTGTGTTCTATATTAACAATGCTCTCAAAAATATTAAAGATATTACTAATTATCGTAAGTACATT
ACCAAAAATACCGATGAGATTATATGTAAATCTATTCTGACTACCAATCTGAAATATCAGTATATGAAATCTGAA
AAAGGGTTTAAACTGCAGAGGAAATTTAAAATTAGCCCGGTTGTTTTTCGTAATGGGAAGGTGATTCTGTATCTC
AATTGTAGCAGCGATTTTTCTACCGATAAGTCTATATATGAGATGCTAAATGATGGTCTGGGGGTGGTGGGTCTA
CAAGTGAAAAATAAATGGACCAATGCAAATGGGAATATTTTCATTGAAAAAGTACTGGATAATACCATTTCTGAC
CCGGGTACTTCTGGTAAGCTAGGTCAGTCTCTAATTGATTACTATATTAATGGTAATCAAAAGTATCGTGTTGAA
AAGTTTACCGATGAAGATAAAAATGCAAAGGTGATTCAGGCTAAGATTAAGAATAAAACCTATAATTATATTCCG
CAAGCTCTGACCCCGGTGATTACCAGGGAATATCTGAGCCATACCGATAAGAAGTTTTCTAAGCAGATTGAGAAT
GTGATTAAAATGGACATGAATTATCGTTATCAGACCCTAAAATCTTTCGTTGAAGATATTGGTGTGATTAAAGAA
CTCAATAATCTGCATTTTAAAAATCAGTATTATACTAATTTTGATTTTATGGGGTTTGAATCTGGTGTTCTGGAA
GAACCGGTACTGATGGGGGCTAACGGTAAAATTAAAGATAAGAAGCAAATTTTTATTAATGGGTTTTTCAAGAAT
CCGAAGGAGAATGTGAAGTTTGGTGTTCTGTATCCGGAAGGGTGTATGGAAAATGCTCAGTCGATTGCTCGTTCT
ATACTAGATTTTGCAACCGCTGGGAAATATAATAAACAAGAGAATAAATATATTAGCAAGAATCTAATGAATATT
GGTTTTAAGCCGTCTGAATGTATTTTCGAATCTTATAAACTCGGTGATATTACCGAATATAAAGCAACCGCTCGT
AAGCTAAAAGAACATGAAAAGGTGGGTTTTGTGATTGCAGTGATTCCGGATATGAATGAACTGGAAGTGGAAAAT
CCGTATAATCCGTTTAAAAAAGTATGGGCAAAACTGAATATTCCGAGCCAGATGATTACCCTGAAAACCACCGAA
AAATTTAAAAATATTGTTGATAAGTCTGGTCTATATTATCTACACAATATTGCTCTCAATATTCTAGGGAAAATT
GGGGGGATTCCGTGGATTATTAAAGATATGCCGGGTAATATTGATTGTTTCATTGGGCTAGATGTGGGTACCCGT
GAAAAAGGTATTCATTTTCCGGCATGTAGCGTTCTATTTGATAAATATGGGAAACTGATTAATTATTATAAGCCG
ACCATTCCGCAGTCTGGTGAGAAAATTGCAGAAACCATTCTGCAAGAGATTTTCGATAATGTTCTGATTAGCTAT
AAAGAAGAAAATGGGGAATATCCGAAAAATATTGTGATTCATCGTGATGGGTTTTCTCGTGAAAATATTGATTGG
TATAAAGAATATTTTGATAAGAAAGGGATTAAATTTAACATTATTGAGGTGAAAAAGAATATTCCGGTTAAAATT
GCAAAGGTGGTTGGGTCGAACATTTGTAATCCCATTAAAGGTTCTTATGTTCTCAAAAATGATAAGGCTTTTATT
GTTACCACCGATATTAAAGATGGGGTTGCTAGCCCGAACCCCCTGAAAATTGAAAAGACCTATGGTGACGTGGAG
ATGAAAAGCATTCTAGAACAGATTTATAGCCTGTCTCAAATTCATGTTGGGAGCACCAAGTCTCTAAGGCTCCCG
ATTACCACCGGTTATGCTGATAAAATTTGCAAAGCAATTGAGTACATTCCGCAAGGTGTTGTTGATAATCGTCTA
TTCTTTCTGtaataacATTGGAAGTGGATAA
[SEQ ID NO: 9]
DNA sequence of codon optimised CbAgo
ATGAATAATCTGACCTTTGAGGCTTTTGAAGGGATTGGTCAACTGAATGAACTGAATTTTTATAAGTATCGTCTG
ATTGGGAAAGGGCAGATTGATAATGTGCATCAAGCTATTTGGTCTGTTAAATATAAACTCCAGGCTAATAATTTT
TTTAAACCGGTGTTTGTGAAAGGTGAGATTCTATATAGCCTAGATGAACTGAAGGTGATTCCGGAATTTGAAAAT
GTTGAGGTGATTCTCGATGGTAATATTATTCTGTCTATTTCTGAGAATACCGATATTTATAAGGATGTGATTGTG
TTCTATATTAACAATGCTCTCAAAAATATTAAAGATATTACTAATTATCGTAAGTACATTACCAAAAATACCGAT
GAGATTATATGTAAATCTATTCTGACTACCAATCTGAAATATCAGTATATGAAATCTGAAAAAGGGTTTAAACTG
CAGAGGAAATTTAAAATTAGCCCGGTTGTTTTTCGTAATGGGAAGGTGATTCTGTATCTCAATTGTAGCAGCGAT
TTTTCTACCGATAAGTCTATATATGAGATGCTAAATGATGGTCTGGGGGTGGTGGGTCTACAAGTGAAAAATAAA
TGGACCAATGCAAATGGGAATATTTTCATTGAAAAAGTACTGGATAATACCATTTCTGACCCGGGTACTTCTGGT
AAGCTAGGTCAGTCTCTAATTGATTACTATATTAATGGTAATCAAAAGTATCGTGTTGAAAAGTTTACCGATGAA
GATAAAAATGCAAAGGTGATTCAGGCTAAGATTAAGAATAAAACCTATAATTATATTCCGCAAGCTCTGACCCCG
GTGATTACCAGGGAATATCTGAGCCATACCGATAAGAAGTTTTCTAAGCAGATTGAGAATGTGATTAAAATGGAC
ATGAATTATCGTTATCAGACCCTAAAATCTTTCGTTGAAGATATTGGTGTGATTAAAGAACTCAATAATCTGCAT
TTTAAAAATCAGTATTATACTAATTTTGATTTTATGGGGTTTGAATCTGGTGTTCTGGAAGAACCGGTACTGATG
GGGGCTAACGGTAAAATTAAAGATAAGAAGCAAATTTTTATTAATGGGTTTTTCAAGAATCCGAAGGAGAATGTG
AAGTTTGGTGTTCTGTATCCGGAAGGGTGTATGGAAAATGCTCAGTCGATTGCTCGTTCTATACTAGATTTTGCA
ACCGCTGGGAAATATAATAAACAAGAGAATAAATATATTAGCAAGAATCTAATGAATATTGGTTTTAAGCCGTCT
GAATGTATTTTCGAATCTTATAAACTCGGTGATATTACCGAATATAAAGCAACCGCTCGTAAGCTAAAAGAACAT
GAAAAGGTGGGTTTTGTGATTGCAGTGATTCCGGATATGAATGAACTGGAAGTGGAAAATCCGTATAATCCGTTT
AAAAAAGTATGGGCAAAACTGAATATTCCGAGCCAGATGATTACCCTGAAAACCACCGAAAAATTTAAAAATATT
GTTGATAAGTCTGGTCTATATTATCTACACAATATTGCTCTCAATATTCTAGGGAAAATTGGGGGGATTCCGTGG
ATTATTAAAGATATGCCGGGTAATATTGATTGTTTCATTGGGCTAGATGTGGGTACCCGTGAAAAAGGTATTCAT
TTTCCGGCATGTAGCGTTCTATTTGATAAATATGGGAAACTGATTAATTATTATAAGCCGACCATTCCGCAGTCT
GGTGAGAAAATTGCAGAAACCATTCTGCAAGAGATTTTCGATAATGTTCTGATTAGCTATAAAGAAGAAAATGGG
GAATATCCGAAAAATATTGTGATTCATCGTGATGGGTTTTCTCGTGAAAATATTGATTGGTATAAAGAATATTTT
GATAAGAAAGGGATTAAATTTAACATTATTGAGGTGAAAAAGAATATTCCGGTTAAAATTGCAAAGGTGGTTGGG
TCGAACATTTGTAATCCCATTAAAGGTTCTTATGTTCTCAAAAATGATAAGGCTTTTATTGTTACCACCGATATT
AAAGATGGGGTTGCTAGCCCGAACCCCCTGAAAATTGAAAAGACCTATGGTGACGTGGAGATGAAAAGCATTCTA
GAACAGATTTATAGCCTGTCTCAAATTCATGTTGGGAGCACCAAGTCTCTAAGGCTCCCGATTACCACCGGTTAT
GCTGATAAAATTTGCAAAGCAATTGAGTACATTCCGCAAGGTGTTGTTGATAATCGTCTATTCTTTCTGtaa

The following examples illustrate the invention:

### EXAMPLES

### Example 1: CbAgo Construct Generation, Expression and Purification

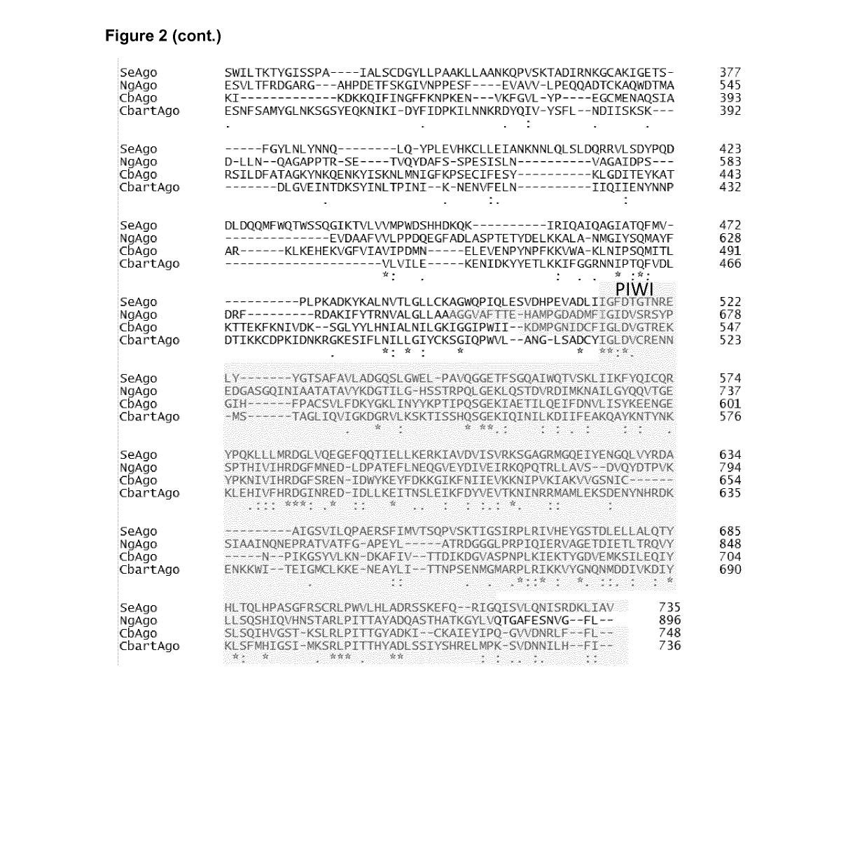

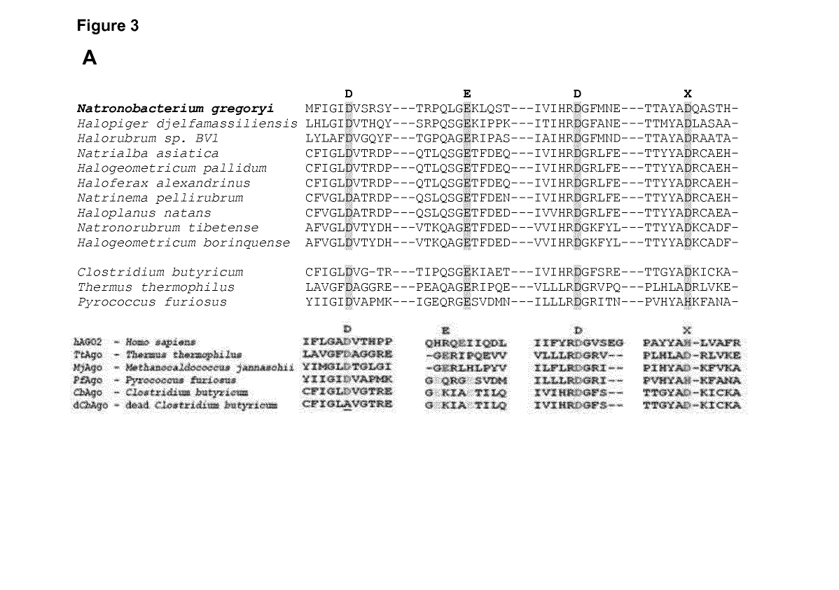

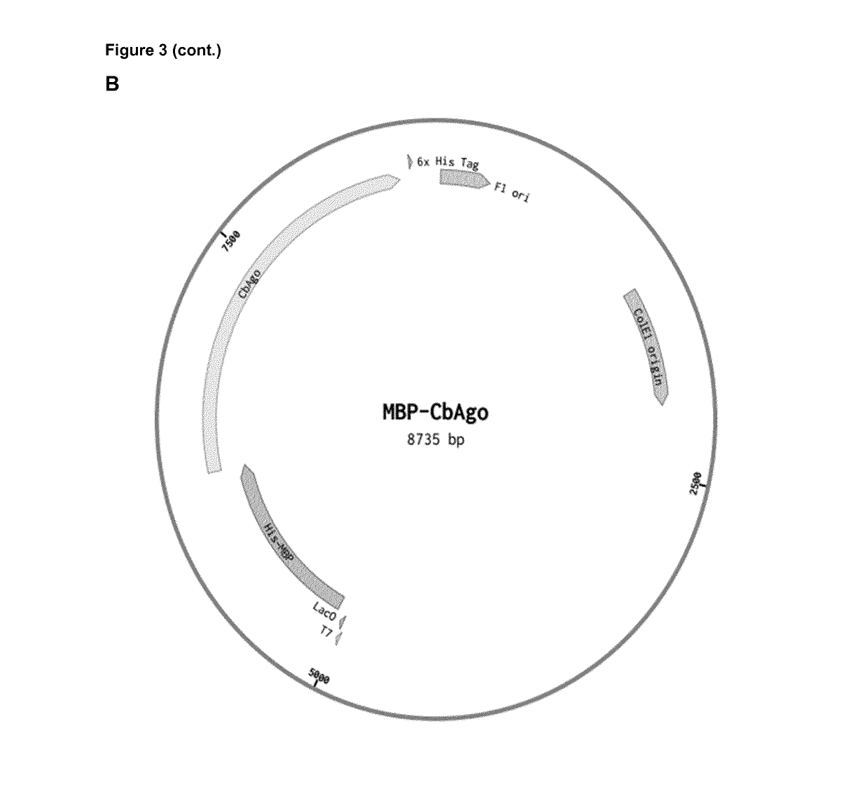

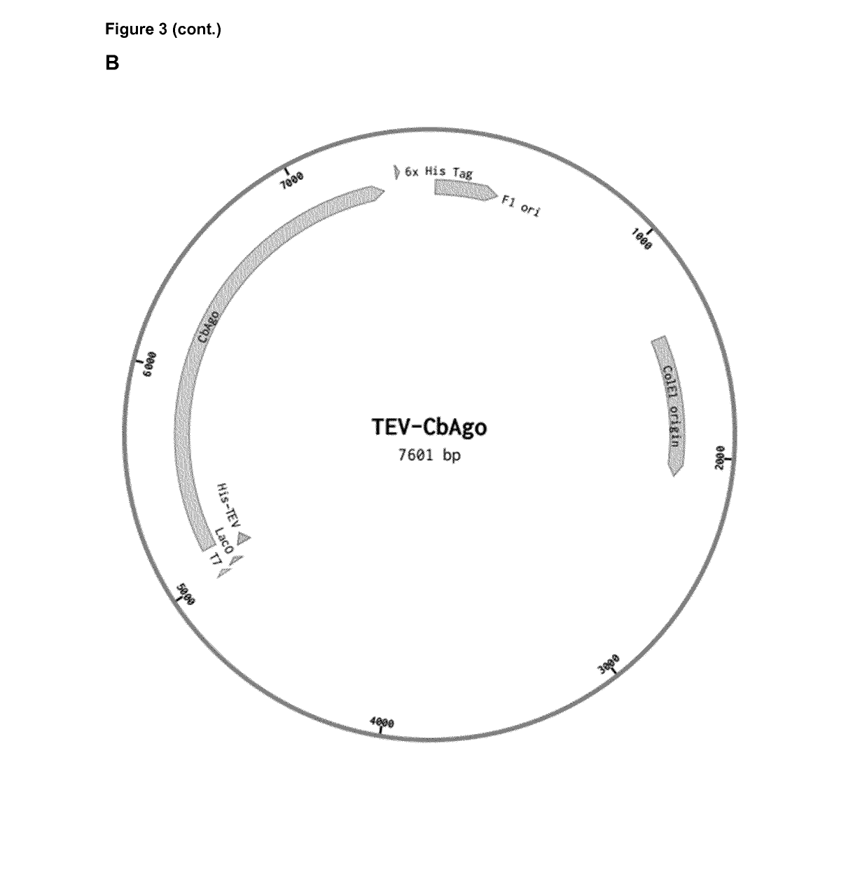

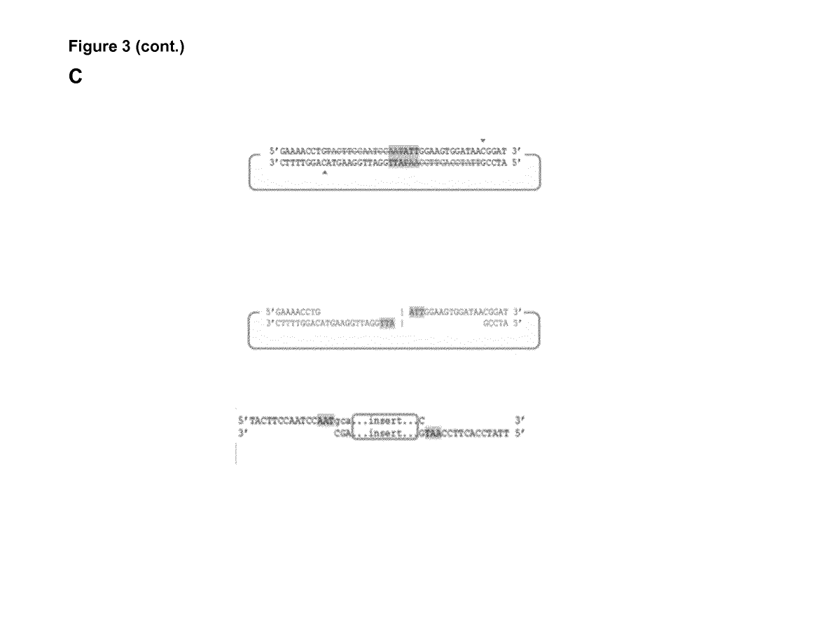

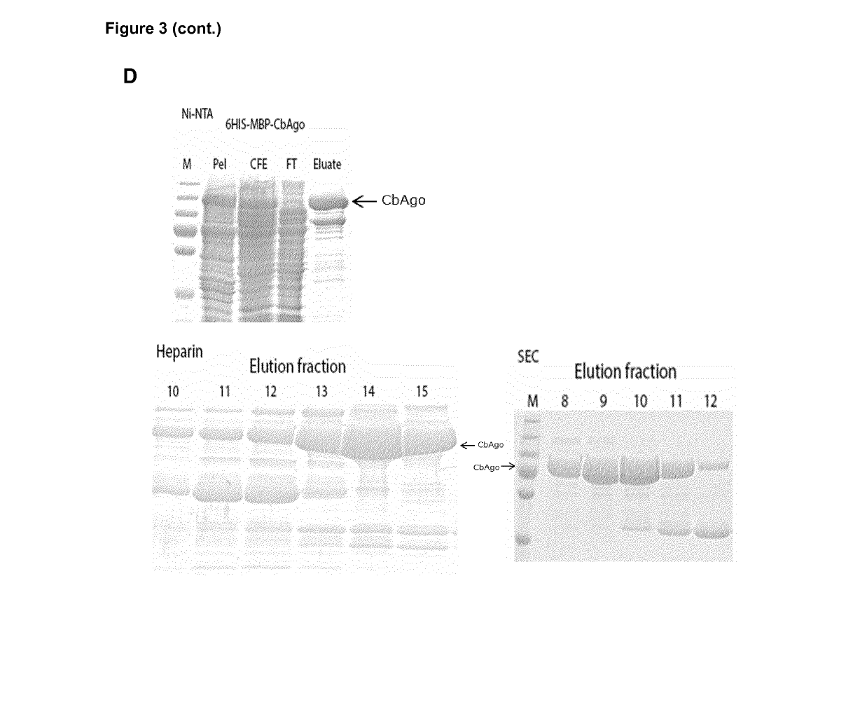

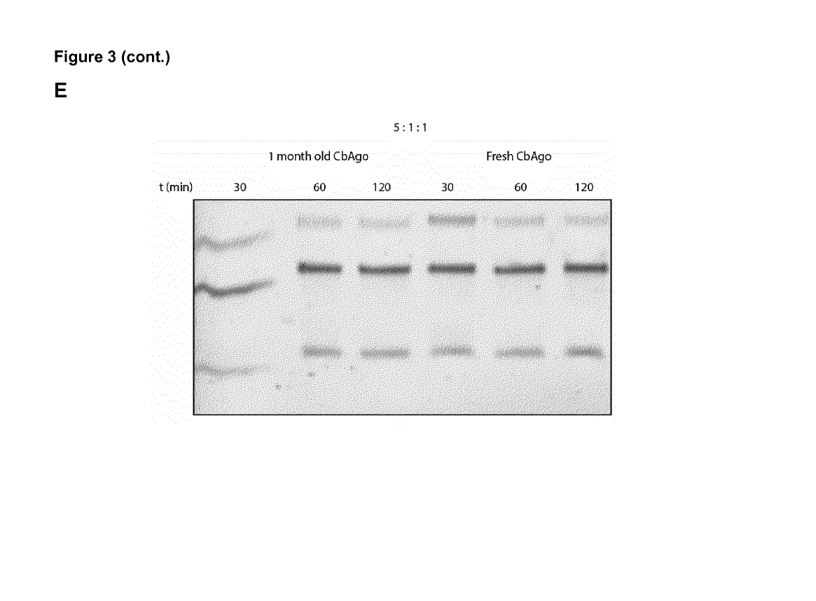

CbAgo was predicted to be a full length Argonaute containing all four domains (FIG. 3A; in SEQ ID NO: 1 DEDX domain residues are italic type and boldface). CbAgo was codon harmonized for E. coli K12 and heterologously expressed and purified in E. coli.

### MBP-CbAgo Construct Generation

pML1-M CbAgo plasmids were generated using Ligation independent cloning (LIC) (FIG. 3B).

Backbone was prepared by mixing pmL 1B (5 μg) or pmL 1M (5 μg), cutsmart buffer (5 μL), SspI (3 μL), MQ water (10 μL) in a reaction volume of 50 μL. The plasmid was cleaned from impurities using a clean and concentrate kit. T4 DNA polymerase and dGTP was used to check back and create sticky/overhangs. This was done by mixing pmL 1B (600 ng) or pmL 1M (600 ng), buffer (3 μL), dGTP (3 μL), 100 mM DTT (1.5 μL) T4 polymerase (0.6 μL) and MQ water (14.7 μL in pmL 1B reaction or 11.4 μL in pmL 1M reaction). A codon optimized CbAgo (SEQ ID NO: 9) insert was generated using PCR polymerase with proof-reading activity to amplify desired insert using LIC flank primers (SEQ ID NO: 8 LIC flanks are underlined) (Phusion polymerase, Thermo). It was purified from NTPs using a PCR clean-up kit. Nucleotide overhangs (15nt) were created using T4 DNA polymerase and dCTP. CbAgo (600 ng), buffer (2 μL), dCTP (2 μL), 100 mM DTT (1 μL) T4 polymerase (0.5 μL) and MQ water (15.9 μL) (FIG. 3C). Vector and insert were then annealed using vector (0.5 μL) and insert (1 μL), which were left at room temperature for 10 min. This was followed by addition of 0.5 μL EDTA (25 mM) and the reaction was left at room temperature for 10 min. The newly formed pML1-M CbAgo construct was transformed in Rosetta (DE3) pLyseS (EMD Millipore) heat-shock competent cells and resulting colonies were checked for desired construct presence in a OneTaq colony PCR using T7 universal primers. Positive colonies were mini-prepped and checked for sequence validity using Sanger sequencing.

### MBP-CbAgo Expression and Purification

4×750 ml Lysogenic Broth (LB) growth media with 50 μg kanamycin/ml LB and 34 μg chloramphenicol in ethanol/ml LB were inoculated with 1 ml overnight culture Rosetta (DE3) pLyseS (EMD Millipore) containing the pML1-M CbAgo expression plasmid and incubated at 37° C. Incubation temperature was set to 20° C. when an O.D. of 0.5 was reached for 30 minutes. IPTG was added to a final concentration of 0.2 mM and culture was incubated at 20° C. overnight allowing protein expression. Cells were harvested and cell pellets were resuspended in 20 mL Cas9 lysis buffer (500 mM NaCl, 20 mM Tris/HCl pH8, 5 mM imidazole, protease inhibitors). Cells were lysed using a sonicator (30%, 5 min, 1 sec on, 2 sec off).

Lysed cells were centrifuged for 45 min at 18 k in SA300 rotor and supernatant was loaded on 2×niNTA superflow column. Column was washed with 45 mL washing buffer (250 mM NaCl, 20 mM Tris/HCl pH8, 20 mM imidazole) and eluted with another 20 mL elution buffer (250 mM NaCl, 20 mM Tris/HCl pH8, 250 mM imidazole). Elution fractions containing protein of expected size were pooled and 25 μL 1M DTT, 750 μl 1.5 mg/mL TEV was added. The pooled fractions were dialysed (12.000-14.000) against 2 L 250 mM KCl, 20 mM HEPES/KOH, 1 mM DTT overnight and diluted in 1:1 10 mM HEPES/KOH pH7.5, and loaded on a heparin FF column pre-equilibrated in IEX-A buffer (150 mM KCl, 20 mM HEPES/KOH pH7.5). Column was washed with 10 mL IEX-A and then with a gradient of IEX-C (2M KCl, 20 mM HEPES/KOH pH7.5). Elution fractions containing protein of expected size were pooled, concentrated and loaded on a hiload 16/600 Superdex 200 column. Elution fractions containing protein of expected size were combined diluted to 5 μM in size exclusion chromatography (SEC) buffer (500 mM KCl, 20 mM HEPES/KOH, 1 mM DTT pH7.5) before flash-freezing in liquid N₂ and storage in −80° C. for activity assays. All figures containing analysed fractions from column purification are displayed in FIG. 3D wherein M=marker, Pel=pellet, CFE=cell free extract, FT=flow through.

CbAgo storage at −80° C. for one month did not impact cleavage ability of CbAgo. Thawed CbAgo, that was stored at −80° C., yielded cleaved DNA in a comparable manner to freshly purified CbAgo (FIG. 3E).

### Example 2: CbAgo Cleaves DNA Targets Using 5′-Phosphorylated DNA Guides and Binds Target RNA/DNA with RNA/DNA Guides

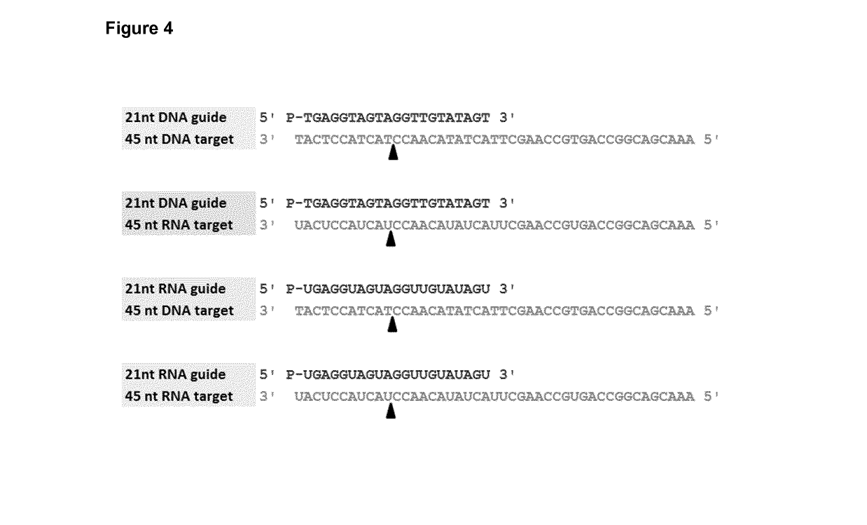

To assess which combinations of RNA/DNA guides and RNA/DNA targets CbAgo was able to cleave, an activity assay was performed with all possible combinations. A molar ratio of 5:1:1 was used for CbAgo:guide:target, which was previously found to yield optimal product formation for PfAgo (Swarts et al., (2015) ibid.). All guides and targets used in this study contain a 5′-phosphate (Table 2; FIG. 4).

TABLE 2
Overview of all guide/target combinations used. All have a 5′-P group.

| | SEQ ID | | |
|---|---|---|---|
| Number | NO: | Sequence (5′ → 3′) | Comment |
| 3466 | 9 | TGAGGTAGTAGGTTGTATAGT | 21 nt Guide; |
| | | | Target is |
| | | | pWUR704 |
| 4017 | 10 | TTATACAACCTACTACCTCGT | 21 nt |
| | | | Guide; |
| | | | Target is |
| | | | pWUR704 |
| 7024 | 11 | UGAGGUAGUAGGUUGUAUAGU | 21 nt Guide; |
| | | | target is |
| | | | pWUR704 |
| 7052 | 12 | UUAUACAACCUACUACCUCGU | 21 nt Guide; |
| | | | target is |
| | | | pWUR704 |
| 7022 | 13 | AAACGACGGCCAGUGCCAAGCUUACUAUACAACCUACUACCUCAU | 45 nt RNA |
| | | | Target; |
| | | | guide is |
| | | | 3466 |
| 7023 | 14 | AAACGACGGCCAGTGCCAAGCTTACTATACAACCTACTACCTCAT | 45 nt DNA |
| | | | target |
| | | | Guide is |
| | | | 3466 |
| 6806 | 15 | TTTCCCAGTCACGACGTTGTAAAACGACGGCCAGTGAATTCACAACGG | 150 nt DNA |
| | | TGAGCAAGTCACTGTTGGCAAGCCAGGATCTGAACAATACCGTCTTGCTTT | target; |
| | | CGAGC | Guide is |
| | | GCTAGCTCTAGAACTAGTCCTCAGCCTAGGCCTCGTTCCGAAGCTG | 6520 |
| 7645 | 16 | AGCAAGTCACTGTTGGCAAGCCAGGATCTGAACAATACCGTCTTG | 45 nt DNA |
| | | | target; |
| | | | Guide is |
| | | | 7647 |
| 7647 | 17 | TAGACGGTATTGTTCAGATCC | 21 nt Guide; |
| | | | Target is |
| | | | 7645 |
| 7760 | 18 | TCCTCAGCCTAGGCCTCGTTCCGAAGCTGTCTTTCGCTGCTGAGG | 45 nt DNA |
| | | | target; |
| | | | Guide is |
| | | | 7761 |
| 7761 | 19 | TTCAGCAGCGAAAGACAGCTT | 21 nt Guide; |
| | | | Target is |
| | | | 7760 |

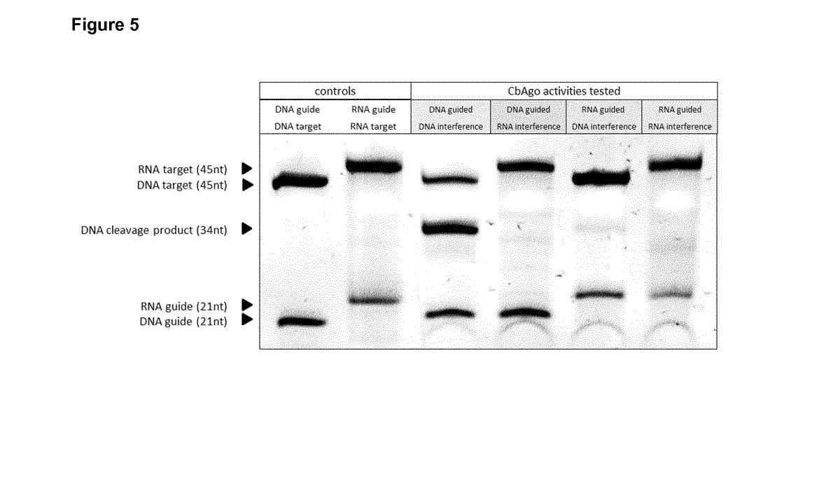

No product bands (34nt) were observed in the DNA and RNA guide/target control assays, which were incubated in the absence of CbAgo, showing that product band formation is a consequence of CbAgo activity. The intensity of the target band is inversely proportional to that of the product band, as product bands are formed from target bands. These gels show that CbAgo could cleave DNA efficiently by utilizing a DNA guide (FIG. 5).

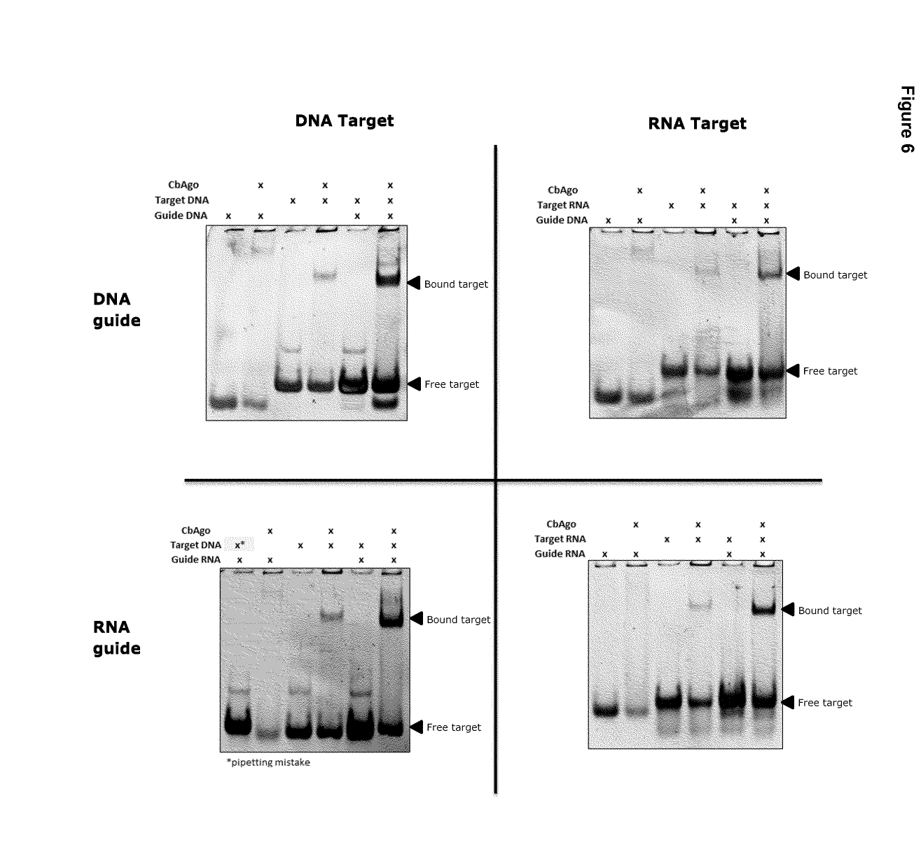

Subsequently, four electrophoretic mobility shift assays (EMSAs) with CbAgo were performed to test its binding capacity to DNA and RNA target and guides (FIG. 6).

CbAgo (10 pmol) was pre-incubated with the desired polynucleotide guide (RNA guide: 7024, DNA guide: 3466, 10 pmol) for 5 min at 37° C. This was followed by addition of a target polynucleotide (RNA target: 7022, DNA guide: 7043, 10 pmol) and another incubation step of 5 min at 37° C. The reactions were then resolved on a 6% Native PAGE gel and stained with SYBR gold.

The EMSA results showed that apo-CbAgo (without guide bound) is able to bind target DNA or RNA to a limited extent. However, binding is much more efficient once CbAgo is preloaded with a complementary DNA or RNA guide. This demonstrates that CbAgo can bind specific RNA and DNA targets when bound to a complementary RNA or DNA guide.

### Example 3: CbAgo Cleaves DNA Targets at a Mesophilic Range of Temperatures

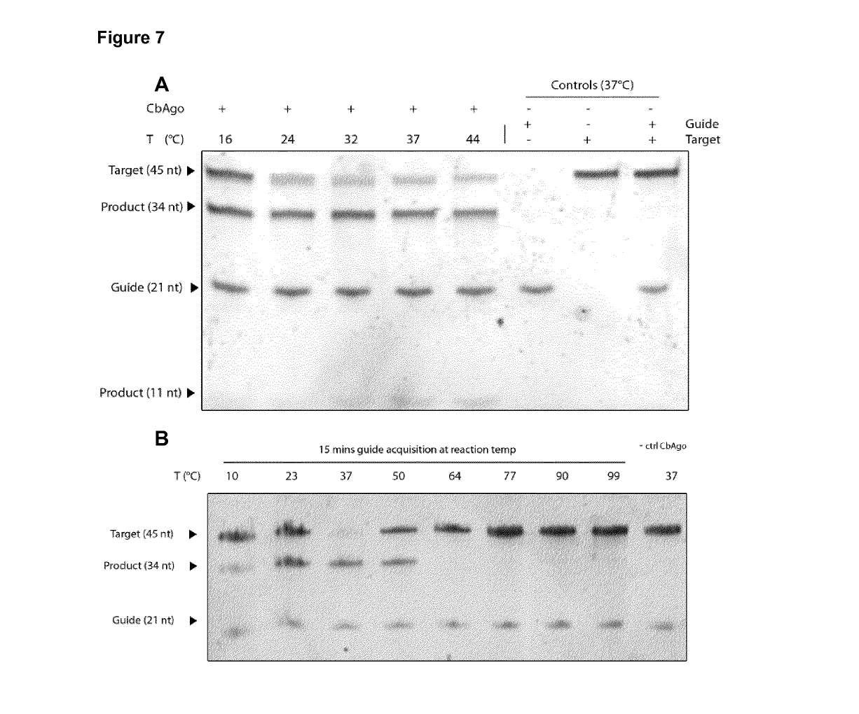

The next activity assays focused on finding the temperature range in which CbAgo exhibits DNA-guided DNA cleavage after one hour (FIG. 7A). CbAgo was observed to be active at a temperature range between at least 10 and 44° C., since 34nt product bands can be observed (FIGS. 7A and 7B). Temperatures of 32° C., 37° C. and 44° C. not only show more degradation of the target bands than lower temperatures, but also showed dim 11 nt product bands. This suggests that CbAgo has more activity in the higher mesophilic temperature spectrum. Control lanes show no product formation for any guide/target combination without adding CbAgo.

In order to determine a temperature limit up to which CbAgo is active, a wider range of temperatures were tested (FIG. 7B). Guides were incubated for 15 minutes at the reaction temperature before adding the target to ensure CbAgo-guide complex formation. This method excludes the possibility of residual CbAgo activity at unintentional lower temperatures when adding the target. CbAgo is active at temperatures as low as 10° C. and as high as 50° C., but most target cleavage occurred at around 37° C.

### Example 4: CbAgo can Use Multiple Cations for Cleavage but has a Preference for Mn²⁺ Cations

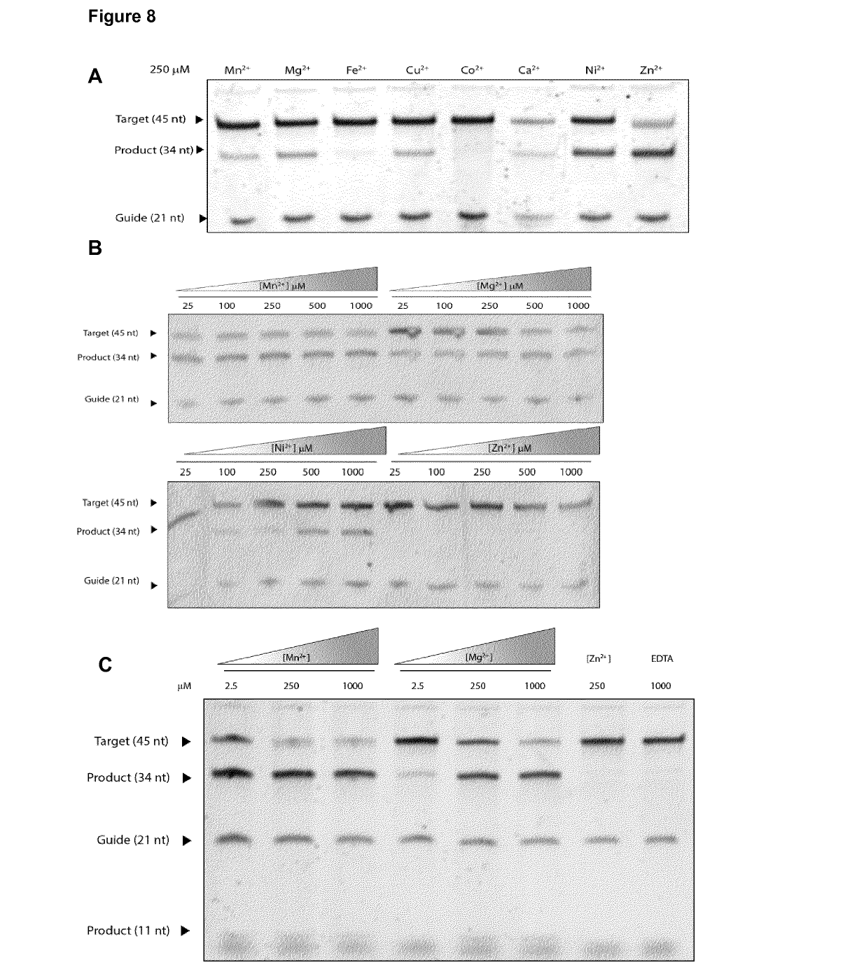

Eight divalent cations were selected and used in an activity assay to determine which cations could mediate CbAgo DNA-guided DNA cleavage (FIG. 8A). CbAgo was observed to use Mn²⁺, Mg²⁺, Fe²⁺, Cu²⁺, Ca²⁺, Ni²⁺ and Zn²⁺ cations based on initial screening, although some cations aided CbAgo cleavage of the target much more efficiently than others, indicated by thicker product bands. Cation concentration in these assays were equal for all cations and thus did not show what cation concentrations limited activity of CbAgo.

Another activity assay was performed in which a range of cation concentrations was used to find the cation threshold at which CbAgo cleaves targets. Four cations were chosen, based on product band strength in the previous cation activity assay. Contrary to the first screening, Zn²⁺ cations did not catalyse CbAgo-guide complex mediated DNA target cleavage. Initial screens with cation concentrations of just 25 μM did not reveal the lower limit at which targets could be cleaved (FIG. 8B). Another assay proved that a 100-fold reduced cation concentration diminished Mg²⁺-catalysed cleavage, but Mn²⁺-catalysed cleavage still was significant (FIG. 8C). This result likely indicates a Mn²⁺ cation preference by CbAgo, since product formation still occurs at very low Mn²⁺ concentrations.

### Example 5: A Single CbAgo-Guide Complex can Cleave Multiple Targets

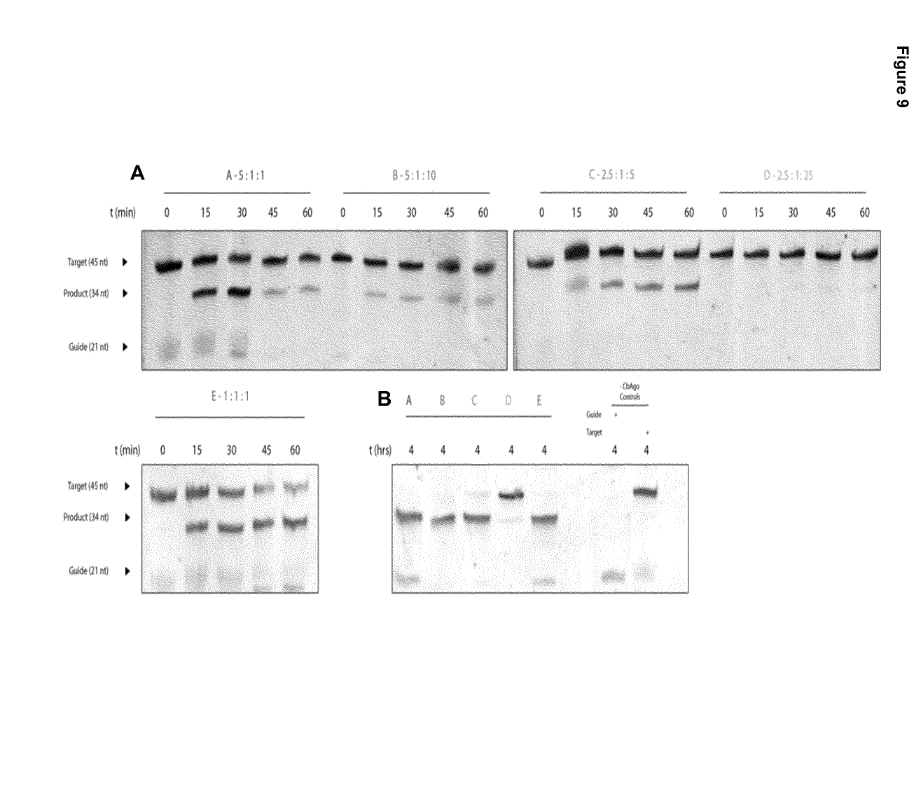

It was noted earlier that CbAgo is able to cleave targets quickly at room temperature but it remained unclear whether a single CbAgo-guide complex could cleave multiple targets. Five reactions with different CbAgo:DNA guide:DNA Target molar ratios were sampled at 6 time points (5 in the span of one hour (FIG. 9A)), one after 4 hours to see complete degradation (FIG. 9B) at 37° C. Samples with an excess of target were diluted to ensure that all samples were loaded on gel with an equal amount of target, to fairly compare product formation between different reaction ratios. The absence of guide bands for diluted samples is expected, as equimolar amounts of guide were added in each reaction.

No product formed when the reaction was stopped immediately after adding the target. Four ratios (A, B, C & E) showed a significant amount of product after all time-points within an hour. For ratio A, bands appear to become fainter after 30 and 60 minutes, suggesting a pipetting mistake since all samples were taken from the same reaction and therefore could never diminish in intensity. After four hours of incubation (FIG. 9B), ratios A & B show complete target degradation, whilst ratios C & E show near complete target degradation. Ratio D, with the biggest excess of target, still shows a strong target band after 4 hours, and only little product. This shows that a 25-fold guide:target excess did not result in complete cleavage of the target band. It was noted that for a successful reaction, guides and CbAgo are both necessary to form the CbAgo complex and these results show ability to cleave a moderate excess of target.

### Example 6: CbAgo Bound ssDNA Guides were Retained with No Detectable Exchange of DNA Guide

In a first attempt to establish CbAgo's ability to retain given guides, a guide competition experiment was designed in which unique guides, with sequence homology to different targets, were added to CbAgo in two acquisition stages of 15 min at 37° C. Once target-complementary guide (target guide) acquisition has taken place and all CbAgo is saturated with target-guide, target non-complementary guides (non-target guide) were added and incubated. When starting the reaction by adding the corresponding target, only target guides bound to CbAgo will result in cleavage of the target, while non-target guides bound to CbAgo result in a decreased or absent cleavage efficiency. A molar ratio CbAgo:guide:target of 3:5:1 was chosen to ensure CbAgo saturation by the provided excess of guides.

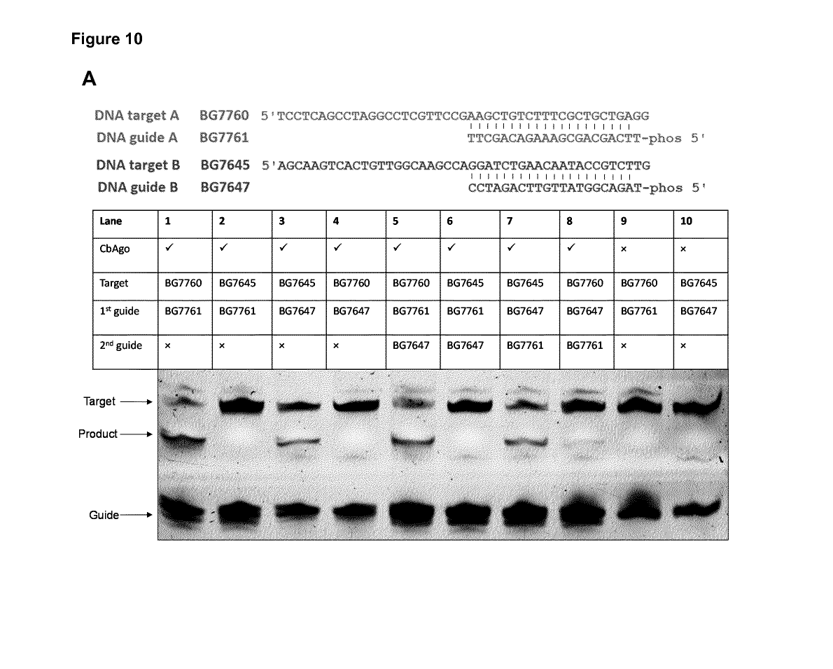

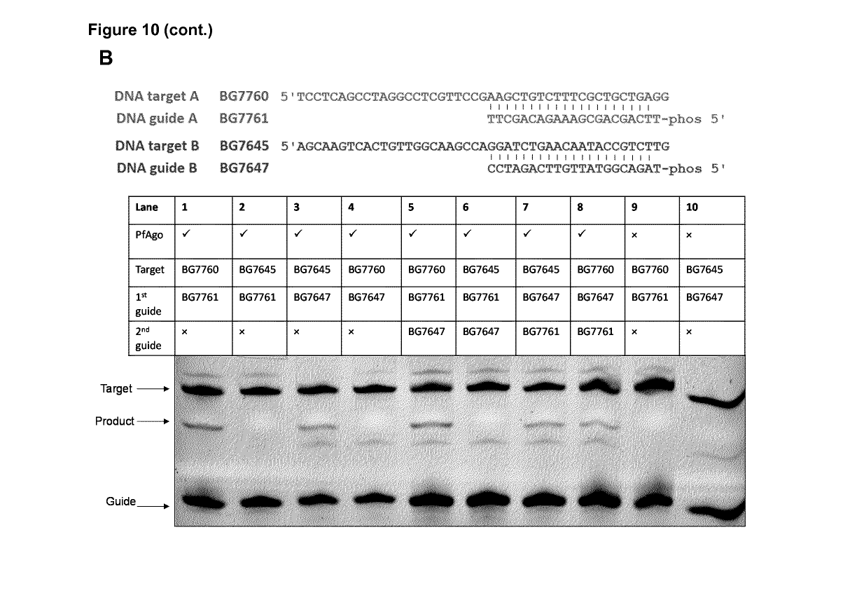

For CbAgo, no cleavage activity was observed when the non-target guide was loaded before the target guide (FIG. 10A; lane 6 and 8). When the target guide was incubated before the non-target guide, activity was observed (FIG. 10A; lane 5 and 7). This suggests that CbAgo retains its original DNA guide.

For PfAgo, cleavage activity was observed when one of the non-target DNA guides was loaded before the target DNA guide (FIG. 10B; Lane 8), suggesting that PfAgo prefers DNA guide 7761 above DNA guide 7747.

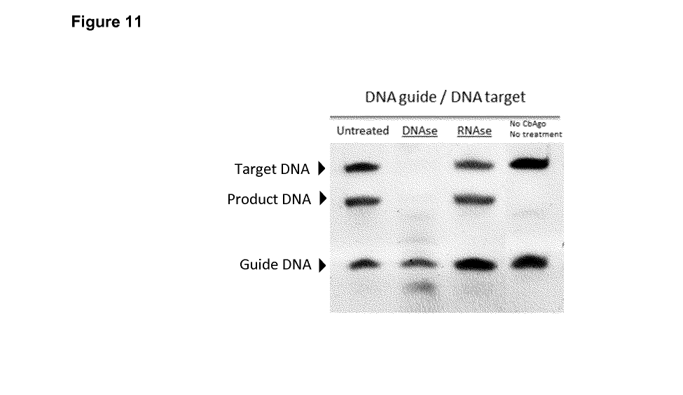

These results were confirmed by DNAse and RNase digestion assays (FIG. 11). These experiments involved a cleavage reaction which was performed using CbAgo, a DNA guide and a complementary ssDNA target. At the end of the reaction (conditions 5 min at 37° C.) the sample was treated with DNAse (lane 2). Whilst the DNA target was degraded the guide was still intact, suggesting that the guide is protected from the endonuclease activity by binding to the CbAgo.

### Example 7: CbAgo Cleaved dsDNA Targets with ssDNA Guides

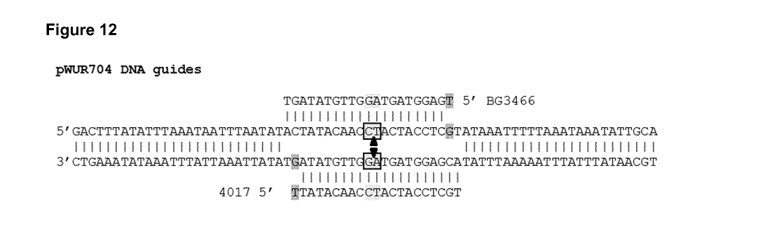

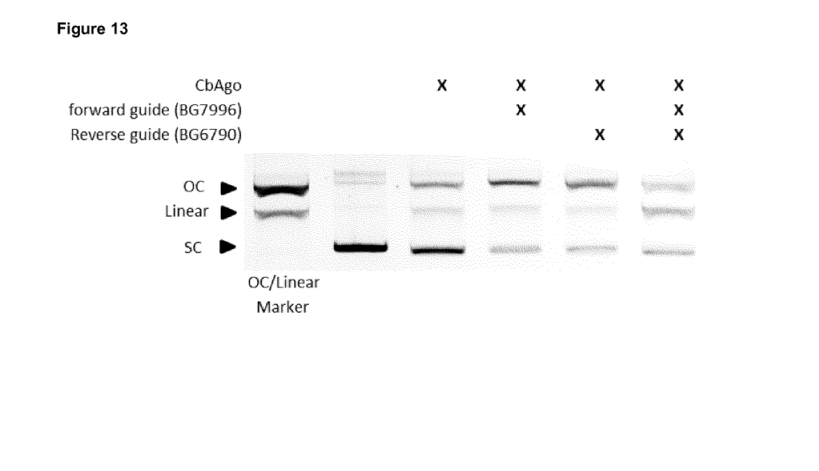

When CbAgo was incubated with ssDNA guides and a supercoiled plasmid there was a shift visible (reaction conditions: 20 mM tris-HCl, 125 mM KCl, 50 μM MnCl, pH 7.5, duration: 16 h). When pWUR704 was incubated with CbAgo enzyme and no DNA guide, minor conversion of the plasmid to open-circularised and linearised DNA was observed (FIG. 13, lane 3). When pWUR704 was incubated with CbAgo and a single DNA guide there was an increase in the proportion of open-circularised plasmid. This demonstrates that CbAgo is able to nick a double stranded DNA target in the presence of a single DNA guide (FIG. 13, lanes 4 and 5). When pWUR704 was incubated with two DNA guides that are substantially complementary to one another, an increase in the amount of linearised plasmid was observed (FIG. 13, lane 6), in comparison to the control assay (with no CbAgo and no DNA guide) and the assays performed with a single DNA guide only. This demonstrates that CbAgo is able to cleave and linearise a dsDNA plasmid by creating two nicks on opposing DNA strands of the plasmid (FIGS. 12 and 13).

1. An isolated prokaryotic Argonaute (pAgo) comprising a PIWI domain having an amino acid sequence of SEQ ID NO:3 or a sequence of at least 50% identity therewith, having binding activity for a single stranded DNA (ssDNA) guide, and having nuclease activity for a target DNA, whereby when a ssDNA guide having substantial complementarity to the target DNA is bound to the pAgo to form a pAgo-guide complex, and when the pAgo-guide complex is associated with the target DNA, there is a site-specific cutting of the target DNA when single stranded, or nicking of the target DNA when double stranded.

2. An isolated pAgo as claimed in claim 1, having an amino acid sequence of SEQ ID NO:1 or a sequence of at least 50% identity therewith; optionally wherein the pAgo has at least 80%, preferably at least 90%, more preferably at least 95% amino acid sequence identity to SEQ ID NO: 1.

3.-7. (canceled)

8. An isolated pAgo as claimed in claim 1, further comprising an N-terminal OB-fold domain; preferably wherein the OB-fold domain comprises SEQ ID NO:2 or sequence of at least 80% identity therewith.

9.-29. (canceled)

30. An in vitro method of modifying a target DNA molecule, comprising the steps of: providing a pAgo of claim 1, and a ssDNA guide, wherein the guide and the pAgo form a pAgo-guide complex; contacting the resulting pAgo-guide complex with the target DNA, the target comprising a nucleotide sequence substantially complementary to the guide sequence, wherein at a specific site the pAgo-guide complex cleaves the target DNA if single stranded, or nicks the target DNA if double stranded.

31. A method of modifying a target DNA molecule in a cell, comprising:
a) providing a pAgo as claimed in claim 1;
b) providing a ssDNA guide; wherein the pAgo and guide form a pAgo-guide complex;
c) introducing the pAgo-guide complex in to a cell; and wherein the guide is substantially complementary to a target DNA comprised in the target DNA, wherein the pAgo-guide complex nicks double stranded target DNA at a specific site.

32. An in vitro method of modifying a target DNA as claimed in claim 30,
wherein the guide and the pAgo form a first pAgo-guide complex; the method further comprising
providing a second pAgo and a second ssDNA guide, wherein the second guide and the second pAgo form a second pAgo-guide complex;
first and second guides having substantial identity to opposed strands of a double stranded-target DNA; and
contacting both first and second pAgo-guide complexes with the double stranded target DNA, wherein the pAgo-guide complexes cleave the double stranded target DNA at a specific site.

33. A method of modifying a double stranded target DNA molecule as claimed in claim 31, wherein
the pAgo and ssDNA guide, form a first pAgo-guide complex; and wherein the method further comprises
providing a second pAgo and a second ssDNA guide, wherein the second guide and the second pAgo form a second pAgo-guide complex; the first and second guides having substantial identity to opposed strands of the double stranded target DNA; and
introducing the pAgo-guide complexes into a cell.

34. A method of modifying a target DNA molecule as claimed in claim 33, wherein an expression vector comprising a DNA sequence of the first pAgo, and optionally the second pAgo, is introduced into the cell separately or simultaneously with the first DNA guide, and optionally the second DNA guide.

35. A method of modifying a target DNA molecule as claimed in claim 34, wherein the cell is comprised in a tissue organ or animal, e.g. human.

36. (canceled)

37. A method of modifying a target DNA molecule as claimed in claim 34, wherein first and second pAgos are encoded on a single expression vector.

38. A method as claimed in claim 33, wherein the cell is a prokaryotic cell.

39. (canceled)

40. A method of modifying a target DNA molecule as claimed in claim 33, wherein the expression vector is comprised in a viral vector e.g. a retroviral or lentiviral vector.

41. (canceled)

42. A method of modifying a target DNA molecule as claimed in claim 33, further comprising providing to the cell a double stranded DNA which inserts at the site of the double stranded break in the chromosomal DNA of the cell.

43. A method of modifying a target DNA molecule as claimed in claim 33, further comprising introducing a mutation in the target DNA resulting in a recombinant DNA, comprising the additional step of introducing a donor template encoding the desired mutation; wherein the mutation is located in the seed region of the pAgo-guide complexes.

44. A method of modifying a target DNA molecule as claimed in claim 33, wherein two site specific double stranded breaks are made resulting in deletion of a DNA-sequence bounded by the breaks.

45.-46. (canceled)

47. A method of modifying a target DNA molecule as claimed in claim 33, wherein the cleavage of the double stranded DNA results in a blunt-end cut.

48.-50. (canceled)

51. A method of modifying a target DNA molecule as claimed in claim 33, wherein the cleavage activity takes place at a temperature between 10 to 50° C., preferably 32 to 44° C., more preferably at 37° C.

52. (canceled)

53. A method of modifying a target DNA molecule as claimed in claim 33, wherein the ssDNA guide is 10 to 50 nucleotides in length, preferably 15 to 30 nucleotides, even more preferably 20 to 25 nucleotides, most preferably 21 nucleotides in length.

54. A method of modifying a target DNA molecule as claimed in 33, wherein the ssDNA guide is not displaceable from a pAgo-guide complex by a subsequently provided or expressed ssDNA guide.

55.-56. (canceled)

57. A method of modifying a target DNA molecule as claimed in claim 33, wherein the target DNA is a supercoiled plasmid or wherein the guide comprises phosphorylated ssDNA.

58.-74. (canceled)


SEQUENCE LISTING (9 sequences)

SEQ ID NO: 1 - protein, 748 aa, Clostridium butyricum
MNNLTFEAFEGIGQLNELNFYKYRLIGKGQIDNVHQAIWSVKYKLQANNFFKPVFVKGEILYSLDELKVIPEFENVEVILDGNIILSISENTDIYKDVIVFYINNALKNIKDITNYRKYITKNTDEIICKSILTTNLKYQYMKSEKGFKLQRKFKISPVVFRNGKVILYLNCSSDFSTDKSIYEMLNDGLGVVGLQVKNKWTNANGNIFIEKVLDNTISDPGTSGKLGQSLIDYYINGNQKYRVEKFTDEDKNAKVIQAKIKNKTYNYIPQALTPVITREYLSHTDKKFSKQIENVIKMDMNYRYQTLKSFVEDIGVIKELNNLHFKNQYYTNFDFMGFESGVLEEPVLMGANGKIKDKKQIFINGFFKNPKENVKFGVLYPEGCMENAQSIARSILDFATAGKYNKQENKYISKNLMNIGFKPSECIFESYKLGDITEYKATARKLKEHEKVGFVIAVIPDMNELEVENPYNPFKKVWAKLNIPSQMITLKTTEKFKNIVDKSGLYYLHNIALNILGKIGGIPWIIKDMPGNIDCFIGLDVGTREKGIHFPACSVLFDKYGKLINYYKPTIPQSGEKIAETILQEIFDNVLISYKEENGEYPKNIVIHRDGFSRENIDWYKEYFDKKGIKFNIIEVKKNIPVKIAKVVGSNICNPIKGSYVLKNDKAFIVTTDIKDGVASPNPLKIEKTYGDVEMKSILEQIYSLSQIHVGSTKSLRLPITTGYADKICKAIEYIPQGVVDNRLFFL

SEQ ID NO: 2 - protein, 149 aa, Clostridium butyricum
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVM

SEQ ID NO: 3 - protein, 221 aa, Clostridium butyricum
KDMPGNIDCFIGLDVGTREKGIHFPACSVLFDKYGKLINYYKPTIPQSGEKIAETILQEIFDNVLISYKEENGEYPKNIVIHRDGFSRENIDWYKEYFDKKGIKFNIIEVKKNIPVKIAKVVGSNICNPIKGSYVLKNDKAFIVTTDIKDGVASPNPLKIEKTYGDVEMKSILEQIYSLSQIHVGSTKSLRLPITTGYADKICKAIEYIPQGVVDNRLFFL

SEQ ID NO: 4 - protein, 896 aa, Natronobacterium gregoryi
MVPKKKRKVATVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 5 - protein, 736 aa, Clostridium bartletti
MVSLDREFNVITEFKNELKPEDIKIFLYSMPIKDINERHSENYAIVQELKKINENPNIVFNEYIIASFNPIINWGKYKDIDVKPDNRNINLDNHTERKILERLLLCDIKNNINNNTTWEQQNKYEIRGNANPAVYLRRPIYSNNNLIIRRKLNFDVNIDKKDIIIGFFLNHEFEYQKTLDEEIKCGNIQKGDKVKDFYNNITYEFLEIAPFSISQENKYMRSSIIEYYLNKGQSYIISGLDKNTKAVLVKNKEGSIFPYIPNRLKKICVFENLGNRRIIEGNKYIKMNPSQNMSESIKLAEGILKNSKYVKFNKANMIVEKIGYKKDIVKRPALKFGKNESNFSAMYGLNKSGSYEQKNIKIDYFIDPKILNNKRDYQIVYSFLNDIISKSKDLGVEINTDKSYINLTPINIKNENVFELNIIQIIENYNNPVLVILEKENIDKYYETLKKIFGGRNNIPTQFVDLDTIKKCDPKIDNKRGKESIFLNILLGIYCKSGIQPWVLANGLSADCYIGLDVCRENNMSTAGLIQVIGKDGRVLKSKTISSHQSGEKIQINILKDIIFEAKQAYKNTYNKKLEHIVFHRDGINREDIDLLKEITNSLEIKFDYVEVTKNINRRMAMLEKSDENYNHRDKENKKWITEIGMCLKKENEAYLITTNPSENMGMARPLRIKKVYGNQNMDDIVKDIYKLSFMHIGSIMKSRLPITTHYADLSSIYSHRELMPKSVDNNILHFI

SEQ ID NO: 6 - protein, 735 aa, Synechococcus elongatus
MDLLSNLRRSSIVLNRFYVKSLSQSDLTAYEYRCIFKKTPELGDEKRLLASICYKLGAIAVRIGSNIITKEAVRPEKLQGHDWQLVQMGTKQLDCRNDAHRCALETFERKFLERDLSASSQTEVRKAAEGGLIWWVVGAKGIEKSGNGWEVHRGRRIDVSLDAEGNLYLEIDIHHRFYTPWTVHQWLEQYPEIPLSYVRNNYLDERHGFINWQYGRFTQERPQDILLDCLGMSLAEYHLNKGATEEEVQQSYVVYVKPISWRKGKLTAHLSRRLSPSLTMEMLAKVAEDSTVCDREKREIRAVFKSIKQSINQRLQEAQKTASWILTKTYGISSPAIALSCDGYLLPAAKLLAANKQPVSKTADIRNKGCAKIGETSFGYLNLYNNQLQYPLEVHKCLLEIANKNNLQLSLDQRRVLSDYPQDDLDQQMFWQTWSSQGIKTVLVVMPWDSHHDKQKIRIQAIQAGIATQFMVPLPKADKYKALNVTLGLLCKAGWQPIQLESVDHPEVADLIIGFDTGTNRELYYGTSAFAVLADGQSLGWELPAVQGGETFSGQAIWQTVSKLIIKFYQICQRYPQKLLLMRDGLVQEGEFQQTIELLKERKIAVDVISVRKSGAGRMGQEIYENGQLVYRDAAIGSVILQPAERSFIMVTSQPVSKTIGSIRPLRIVHEYGSTDLELLALQTYHLTQLHPASGFRSCRLPWVLHLADRSSKEFQRIGQISVLQNISRDKLIAV

SEQ ID NO: 7 - DNA, 3961 bp, Artificial Sequence
  DNA Sequence of pWUR704
gtcgactttatatttaaataatttaatatactatacaacctactacctcgtataaatttttaaataaatattgcattcaagcttttaatttaattaaatggccgctctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctccgtgtcgttctgtccactcctgaatcccattccagaaattctctagcgattccagaagtttctcagagtcggaaagttgaccagacattacgaactggcacagatggtcataacctgaaggaagatctctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgcctgctgctccatacaagccaaccacggcctccagaagaagatgttggcgacctcgtataggggatctccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcagatgaaatcacgccatgtagtgtattgaccgattccttgcggtccgaatgggccgaacccgctcgtctggctaagatcggccgcagcgatcgcatccataacctccgcgaccggttgcagaacagcgggcagttcggtttcaggcaggtcttgcaacgtgacaccctgtgcacggcgggagatgcaataggtcaggctctcgctaaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgccgaacttttcgatcagaaacttctcaacagacgtcgcggtgagttcaggctttttcatgtgcctcacacctccttaagggtcgtgggcgggaacccgagacgggcgagttgccgcgtttcctctccgcccaggtccgcccggtgcggggaaaaccccccaaaaggagccctttttccccgcatccggcgctatcgtaaaaacctcacgcgcccttgtcaaacggtcgggccttaaggtttctgttatactccccccggggatcgatccccggcccgacgggagccgggcggtggtggcctgggctagc

SEQ ID NO: 8 - DNA, 2281 bp, Artificial Sequence
  DNA sequence of codon optimised CbAgo with LIC flanks
tacttccaatccaatgcaaataatctgacctttgaggcttttgaagggattggtcaactgaatgaactgaatttttataagtatcgtctgattgggaaagggcagattgataatgtgcatcaagctatttggtctgttaaatataaactccaggctaataatttttttaaaccggtgtttgtgaaaggtgagattctatatagcctagatgaactgaaggtgattccggaatttgaaaatgttgaggtgattctcgatggtaatattattctgtctatttctgagaataccgatatttataaggatgtgattgtgttctatattaacaatgctctcaaaaatattaaagatattactaattatcgtaagtacattaccaaaaataccgatgagattatatgtaaatctattctgactaccaatctgaaatatcagtatatgaaatctgaaaaagggtttaaactgcagaggaaatttaaaattagcccggttgtttttcgtaatgggaaggtgattctgtatctcaattgtagcagcgatttttctaccgataagtctatatatgagatgctaaatgatggtctgggggtggtgggtctacaagtgaaaaataaatggaccaatgcaaatgggaatattttcattgaaaaagtactggataataccatttctgacccgggtacttctggtaagctaggtcagtctctaattgattactatattaatggtaatcaaaagtatcgtgttgaaaagtttaccgatgaagataaaaatgcaaaggtgattcaggctaagattaagaataaaacctataattatattccgcaagctctgaccccggtgattaccagggaatatctgagccataccgataagaagttttctaagcagattgagaatgtgattaaaatggacatgaattatcgttatcagaccctaaaatctttcgttgaagatattggtgtgattaaagaactcaataatctgcattttaaaaatcagtattatactaattttgattttatggggtttgaatctggtgttctggaagaaccggtactgatgggggctaacggtaaaattaaagataagaagcaaatttttattaatgggtttttcaagaatccgaaggagaatgtgaagtttggtgttctgtatccggaagggtgtatggaaaatgctcagtcgattgctcgttctatactagattttgcaaccgctgggaaatataataaacaagagaataaatatattagcaagaatctaatgaatattggttttaagccgtctgaatgtattttcgaatcttataaactcggtgatattaccgaatataaagcaaccgctcgtaagctaaaagaacatgaaaaggtgggttttgtgattgcagtgattccggatatgaatgaactggaagtggaaaatccgtataatccgtttaaaaaagtatgggcaaaactgaatattccgagccagatgattaccctgaaaaccaccgaaaaatttaaaaatattgttgataagtctggtctatattatctacacaatattgctctcaatattctagggaaaattggggggattccgtggattattaaagatatgccgggtaatattgattgtttcattgggctagatgtgggtacccgtgaaaaaggtattcattttccggcatgtagcgttctatttgataaatatgggaaactgattaattattataagccgaccattccgcagtctggtgagaaaattgcagaaaccattctgcaagagattttcgataatgttctgattagctataaagaagaaaatggggaatatccgaaaaatattgtgattcatcgtgatgggttttctcgtgaaaatattgattggtataaagaatattttgataagaaagggattaaatttaacattattgaggtgaaaaagaatattccggttaaaattgcaaaggtggttgggtcgaacatttgtaatcccattaaaggttcttatgttctcaaaaatgataaggcttttattgttaccaccgatattaaagatggggttgctagcccgaaccccctgaaaattgaaaagacctatggtgacgtggagatgaaaagcattctagaacagatttatagcctgtctcaaattcatgttgggagcaccaagtctctaaggctcccgattaccaccggttatgctgataaaatttgcaaagcaattgagtacattccgcaaggtgttgttgataatcgtctattctttctgtaataacattggaagtggataa

SEQ ID NO: 9 - DNA, 2247 bp, Artificial Sequence
  DNA sequence of codon optimised CbAgo
atgaataatctgacctttgaggcttttgaagggattggtcaactgaatgaactgaatttttataagtatcgtctgattgggaaagggcagattgataatgtgcatcaagctatttggtctgttaaatataaactccaggctaataatttttttaaaccggtgtttgtgaaaggtgagattctatatagcctagatgaactgaaggtgattccggaatttgaaaatgttgaggtgattctcgatggtaatattattctgtctatttctgagaataccgatatttataaggatgtgattgtgttctatattaacaatgctctcaaaaatattaaagatattactaattatcgtaagtacattaccaaaaataccgatgagattatatgtaaatctattctgactaccaatctgaaatatcagtatatgaaatctgaaaaagggtttaaactgcagaggaaatttaaaattagcccggttgtttttcgtaatgggaaggtgattctgtatctcaattgtagcagcgatttttctaccgataagtctatatatgagatgctaaatgatggtctgggggtggtgggtctacaagtgaaaaataaatggaccaatgcaaatgggaatattttcattgaaaaagtactggataataccatttctgacccgggtacttctggtaagctaggtcagtctctaattgattactatattaatggtaatcaaaagtatcgtgttgaaaagtttaccgatgaagataaaaatgcaaaggtgattcaggctaagattaagaataaaacctataattatattccgcaagctctgaccccggtgattaccagggaatatctgagccataccgataagaagttttctaagcagattgagaatgtgattaaaatggacatgaattatcgttatcagaccctaaaatctttcgttgaagatattggtgtgattaaagaactcaataatctgcattttaaaaatcagtattatactaattttgattttatggggtttgaatctggtgttctggaagaaccggtactgatgggggctaacggtaaaattaaagataagaagcaaatttttattaatgggtttttcaagaatccgaaggagaatgtgaagtttggtgttctgtatccggaagggtgtatggaaaatgctcagtcgattgctcgttctatactagattttgcaaccgctgggaaatataataaacaagagaataaatatattagcaagaatctaatgaatattggttttaagccgtctgaatgtattttcgaatcttataaactcggtgatattaccgaatataaagcaaccgctcgtaagctaaaagaacatgaaaaggtgggttttgtgattgcagtgattccggatatgaatgaactggaagtggaaaatccgtataatccgtttaaaaaagtatgggcaaaactgaatattccgagccagatgattaccctgaaaaccaccgaaaaatttaaaaatattgttgataagtctggtctatattatctacacaatattgctctcaatattctagggaaaattggggggattccgtggattattaaagatatgccgggtaatattgattgtttcattgggctagatgtgggtacccgtgaaaaaggtattcattttccggcatgtagcgttctatttgataaatatgggaaactgattaattattataagccgaccattccgcagtctggtgagaaaattgcagaaaccattctgcaagagattttcgataatgttctgattagctataaagaagaaaatggggaatatccgaaaaatattgtgattcatcgtgatgggttttctcgtgaaaatattgattggtataaagaatattttgataagaaagggattaaatttaacattattgaggtgaaaaagaatattccggttaaaattgcaaaggtggttgggtcgaacatttgtaatcccattaaaggttcttatgttctcaaaaatgataaggcttttattgttaccaccgatattaaagatggggttgctagcccgaaccccctgaaaattgaaaagacctatggtgacgtggagatgaaaagcattctagaacagatttatagcctgtctcaaattcatgttgggagcaccaagtctctaaggctcccgattaccaccggttatgctgataaaatttgcaaagcaattgagtacattccgcaaggtgttgttgataatcgtctattctttctgtaa